(19) United States
(12) Patent Application Publication
(10) Pub. No.: US 2018/0078156 A1
(43) Pub. Date: Mar. 22, 2018

(54) Blood Pressure Measurement Data Processing Method and Apparatus

(71) Applicant: Huawei Technologies Co., Ltd., Shenzhen (CN)

(72) Inventors: Wenjuan Chen, Shenzhen (CN); Honggang Li, Shenzhen (CN)

(21) Appl. No.: 15/544,427

(22) PCT Filed: Aug. 14, 2015
(86) PCT No.: PCT/CN2015/086966

§ 371 Date: Jul. 18, 2017

(51) Int. Cl. A61B 5/021; A61B 5/117; A61B 5/0402; A61B 5/00

(52) U.S. Cl. CPC A61B 5/02125; A61B 5/117; A61B 2560/0238; A61B 5/7278; A61B 5/0402

(57) ABSTRACT

A blood pressure measurement data processing method includes obtaining first calibration data of a user and pre-stored second calibration data of the user; determining, according to the first calibration data and the second calibration data, an optimal function used to represent a function relationship between a pulse wave transmission time and a blood pressure value of the user; obtaining a current pulse wave transmission time of the user; and calculating a current blood pressure value of the user according to the current pulse wave transmission time and the optimal function.

### CROSS-REFERENCE TO RELATED APPLICATION

This application is a national stage of International Application No. PCT/CN2015/086966, filed on Aug. 14, 2015, which is hereby incorporated by reference in its entirety.

### TECHNICAL FIELD

The present disclosure relates to a blood pressure measurement data processing method and an apparatus.

### BACKGROUND

In recent years, with development of mobile medical technologies, it is more convenient to monitor blood pressure. A commonly used cuff-less blood pressure measurement method is to determine blood pressure based on a relationship between the blood pressure and a pulse wave velocity. When the blood pressure increases, a blood vessel dilates, and the pulse wave velocity accelerates. Otherwise, the pulse wave velocity slows. Generally, the pulse wave velocity is indirectly represented by using a pulse wave transmission time (PTT). Existing research shows that blood pressure and PTT have a quasi-linear relationship. However, because physiological parameters of persons such as arterial wall elasticity and blood density are different, a relationship between the PTT and blood pressure of a measured object is object dependent, and calibration needs to be performed for each measured object before the blood pressure is calculated by using the PTT.

In the prior art, diastolic blood pressure and systolic blood pressure are usually measured by using a conventional sphygmomanometer, and a measurement result is transmitted to a blood pressure measurement apparatus. When a user wears a cuff-less blood pressure measurement apparatus, a microprocessor module calculates a calibration parameter according to the measurement result of the conventional sphygmomanometer and a PTT value determined by the cuff-less blood pressure measurement apparatus, so as to determine a blood pressure calculation policy.

However, in such a manner, when the user performs calibration again, original calibration data cannot be used again. Consequently, data is wasted, and calibration is not precise enough.

### SUMMARY

A technical problem to be mainly resolved by this application is how to make calibration more accurate when a user wears a cuff-less blood pressure measurement apparatus.

In view of this, this application provides a blood pressure measurement data processing method and an apparatus. When different users wear a cuff-less blood pressure measurement apparatus, an optimal function between a pulse wave transmission time and a blood pressure value of a user can be determined by using pre-stored calibration data and with reference to calibration data generated when the user performs manual calibration before performing measurement using the cuff-less blood pressure measurement apparatus, so that the original calibration data can be fully used, and calibration is more accurate.

According to a first aspect, this application provides a blood pressure measurement data processing method, and the method includes obtaining, by a cuff-less blood pressure measurement apparatus, first calibration data of a user, where the first calibration data is data generated when the user performs a manual calibration process before measuring blood pressure by using the cuff-less blood pressure measurement apparatus; obtaining pre-stored second calibration data of the user; determining, according to the first calibration data and the second calibration data, an optimal function used to represent a relationship between a pulse wave transmission time and a blood pressure value of the user; obtaining a current pulse wave transmission time of the user; and calculating a current blood pressure value of the user according to the current pulse wave transmission time and the optimal function.

With reference to the first aspect, in a first possible implementation of the first aspect, the determining, according to the first calibration data and the second calibration data, an optimal function used to represent a relationship between a pulse wave transmission time and a blood pressure value of the user includes determining a first function according to the first calibration data, determining a second function according to the second calibration data, determining a degree of difference between the first function and the second function, and determining the optimal function according to the degree of difference.

With reference to the first possible implementation of the first aspect, in a second possible implementation of the first aspect, the determining a first function according to the first calibration data includes determining the first function according to the first calibration data by using a least square method, and the determining a second function according to the second calibration data includes determining the second function according to the second calibration data by using the least square method.

With reference to the first possible implementation of the first aspect, in a third possible implementation of the first aspect, the determining the optimal function according to the degree of difference includes, if the degree of difference is less than a first preset threshold, using a third function as the optimal function, where the third function is determined by using the first calibration data and the second calibration data.

With reference to the third possible implementation of the first aspect, in a fourth possible implementation of the first aspect, the determining the optimal function according to the degree of difference further includes using the second function as the optimal function if the degree of difference is greater than the first preset threshold and a quantity of samples of the first calibration data is less than a second preset threshold, or using the first function as the optimal function if the degree of difference is greater than the first preset threshold and a quantity of samples of the first calibration data is greater than the second preset threshold.

With reference to the third possible implementation of the first aspect, in a fifth possible implementation of the first aspect, the determining the optimal function according to the degree of difference further includes using the third function as the optimal function if the degree of difference is greater than the first preset threshold and a quantity of samples of the first calibration data is less than a second preset threshold, where the third function is determined by using a combination of the first calibration data and the second calibration data; or removing an abnormal data point from the first calibration data and using a fourth function as the optimal function if the degree of difference is greater than the first preset threshold and a quantity of samples of the first calibration data is greater than the second preset threshold, where the fourth function is calculated by using a combination of the second calibration data and remaining first calibration data.

With reference to the first aspect, in a sixth possible implementation of the first aspect, the determining, according to the first calibration data and the second calibration data, an optimal function of a relationship between a pulse wave transmission time and a blood pressure value of the user includes determining a first function according to the first calibration data, determining a third function according to a combination of the first calibration data and the second calibration data, determining a degree of difference between the first function and the third function, and determining the optimal function according to the degree of difference.

With reference to the sixth possible implementation of the first aspect, in a seventh possible implementation of the first aspect, the determining a first function according to the first calibration data includes determining the first function according to the first calibration data by using a least square method, and the determining a third function according to a combination of the first calibration data and the second calibration data includes determining the third function according to the combination of the first calibration data and the second calibration data by using the least square method.

With reference to the sixth possible implementation of the first aspect, in an eighth possible implementation of the first aspect, the determining the optimal function according to the degree of difference includes using the third function as the optimal function if the degree of difference is less than a first preset threshold.

With reference to the eighth possible implementation of the first aspect, in a ninth possible implementation of the first aspect, the determining the optimal function according to the degree of difference further includes using a second function as the optimal function if the degree of difference is greater than the first preset threshold and a quantity of samples of the first calibration data is less than a second preset threshold, where the second function is determined by using the second calibration data; or using the first function as the optimal function if the degree of difference is greater than the first preset threshold and a quantity of samples of the first calibration data is greater than the second preset threshold.

With reference to the eighth possible implementation of the first aspect, in a tenth possible implementation of the first aspect, the determining the optimal function according to the degree of difference further includes using the third function as the optimal function if the degree of difference is greater than the first preset threshold and a quantity of samples of the first calibration data is less than a second preset threshold; or removing an abnormal data point from the first calibration data and using a fourth function as the optimal function if the degree of difference is greater than the first preset threshold and a quantity of samples of the first calibration data is greater than the second preset threshold, where the fourth function is calculated by using a combination of the second calibration data and remaining first calibration data.

With reference to the first aspect, in an eleventh possible implementation of the first aspect, the first calibration data and the second calibration data separately include at least one group of a blood pressure value and a corresponding pulse wave transmission time, and the determining, according to the first calibration data and the second calibration data, an optimal function used to represent a relationship between a pulse wave transmission time and a blood pressure value of the user includes obtaining a current pulse wave transmission time of the user; selecting, from the first calibration data and the second calibration data, a pulse wave transmission time closest to the current pulse wave transmission time; using calibration data in which the pulse wave transmission time closest to the current pulse wave transmission time exists as optimal calibration data; and using a function as the optimal function, where the function is determined according to the optimal calibration data.

With reference to the first aspect, in a twelfth possible implementation of the first aspect, the obtaining pre-stored second calibration data of the user includes obtaining, by the cuff-less blood pressure measurement apparatus, an identity of the user; and obtaining the second calibration data from multiple pieces of pre-stored calibration data according to the identity of the user.

With reference to the twelfth possible implementation of the first aspect, in a thirteenth possible implementation of the first aspect, the obtaining an identity of the user includes determining the identity of the user according to at least one of a first electrocardiosignal or a first pulse wave signal in the first calibration data of the user, or determining the identity of the user according to at least one of a current electrocardiosignal or a current pulse wave signal generated when the user currently performs measurement by using the cuff-less blood pressure measurement apparatus.

With reference to the first aspect, in a fourteenth possible implementation of the first aspect, the obtaining a current pulse wave transmission time of the user and calculating a current blood pressure value of the user according to the current pulse wave transmission time and the optimal function includes obtaining a current electrocardiosignal and a current pulse wave signal generated when the user currently performs measurement by using the cuff-less blood pressure measurement apparatus, calculating the current pulse wave transmission time, and calculating the current blood pressure value of the user according to the optimal function and the current pulse wave transmission time.

According to a second aspect, a cuff-less blood pressure measurement apparatus is provided, and the cuff-less blood pressure measurement apparatus includes a first obtaining module, a second obtaining module, a determining module, and a calculation module, where the first obtaining module is configured to obtain first calibration data, where the first calibration data is data generated when a user performs a manual calibration process before measuring blood pressure by using the cuff-less blood pressure measurement apparatus; the second obtaining module is configured to obtain pre-stored second calibration data of the user; the determining module is configured to determine, according to the first calibration data and the second calibration data, an optimal function used to represent a relationship between a pulse wave transmission time and a blood pressure value of the user; and the calculation module is configured to obtain a current pulse wave transmission time of the user and calculate a current blood pressure value of the user according to the current pulse wave transmission time and the optimal function.

With reference to the second aspect, in a first possible implementation of the second aspect, the determining module includes a first determining unit, a second determining unit, a third determining unit, and a fourth determining unit, where the first determining unit is configured to determine a first function according to the first calibration data, the second determining unit is configured to determine a second function according to the second calibration data, the third determining unit is configured to determine a degree of difference between the first function and the second function, and the fourth determining unit is configured to determine the optimal function according to the degree of difference.

With reference to the first possible implementation of the second aspect, in a second possible implementation of the second aspect, the first determining unit is configured to determine the first function according to the first calibration data by using a least square method, and the second determining unit is configured to determine the second function according to the second calibration data by using the least square method.

With reference to the second possible implementation of the second aspect, in a third possible implementation of the second aspect, the fourth determining unit is configured to use a third function as the optimal function when the degree of difference is less than a first preset threshold, where the third function is determined by using the first calibration data and the second calibration data.

With reference to the third possible implementation of the second aspect, in a fourth possible implementation of the second aspect, the fourth determining unit is configured to use the second function as the optimal function when the degree of difference is greater than the first preset threshold and a quantity of samples of the first calibration data is less than a second preset threshold, or the fourth determining unit is configured to use the first function as the optimal function when the degree of difference is greater than the first preset threshold and a quantity of samples of the first calibration data is greater than the second preset threshold.

With reference to the third possible implementation of the second aspect, in a fifth possible implementation of the second aspect, the fourth determining unit is configured to use the third function as the optimal function when the degree of difference is greater than the first preset threshold and a quantity of samples of the first calibration data is less than a second preset threshold, where the third function is determined by using the first calibration data and the second calibration data; or the fourth determining unit is configured to remove an abnormal data point from the first calibration data and use a fourth function as the optimal function when the degree of difference is greater than the first preset threshold and a quantity of samples of the first calibration data is greater than the second preset threshold, where the fourth function is calculated by using a combination of the second calibration data and remaining first calibration data.

With reference to the second aspect, in a sixth possible implementation of the second aspect, the determining module includes a first determining unit, a second determining unit, a third determining unit, and a fourth determining unit, where the first determining unit is configured to determine a first function according to the first calibration data, the second determining unit is configured to determine a third function according to a combination of the first calibration data and the second calibration data, the third determining unit is configured to determine a degree of difference between the first function and the third function, and the fourth determining unit is configured to determine the optimal function according to the degree of difference.

With reference to the sixth possible implementation of the second aspect, in a seventh possible implementation of the second aspect, the first determining unit is configured to determine the first function according to the first calibration data by using a least square method, and the second determining unit is configured to determine a second function according to the second calibration data by using the least square method.

With reference to the sixth possible implementation of the second aspect, in an eighth possible implementation of the second aspect, the fourth determining unit is configured to use the third function as the optimal function when the degree of difference is less than a first preset threshold.

With reference to the eighth possible implementation of the second aspect, in a ninth possible implementation of the second aspect, the fourth determining unit is configured to use a second function as the optimal function when the degree of difference is greater than the first preset threshold and a quantity of samples of the first calibration data is less than a second preset threshold, where the second function is determined by using the second calibration data; or the fourth determining unit is configured to use the first function as the optimal function when the degree of difference is greater than the first preset threshold and a quantity of samples of the first calibration data is greater than the second preset threshold.

With reference to the eighth possible implementation of the second aspect, in a tenth possible implementation of the second aspect, the fourth determining unit is configured to use the third function as the optimal function when the degree of difference is greater than the first preset threshold and a quantity of samples of the first calibration data is less than a second preset threshold; or the fourth determining unit is configured to remove an abnormal data point from the first calibration data and use a fourth function as the optimal function when the degree of difference is greater than the first preset threshold and a quantity of samples of the first calibration data is greater than the second preset threshold, where the fourth function is calculated by using a combination of the second calibration data and remaining first calibration data.

With reference to the second aspect, in an eleventh possible implementation of the second aspect, the first calibration data and the second calibration data separately include at least one group of a blood pressure value and a corresponding pulse wave transmission time, and the determining module includes an obtaining unit, a selection unit, and a determining unit, where the obtaining unit is configured to obtain a current pulse wave transmission time of the user; the selection unit is configured to select, from the first calibration data and the second calibration data, a pulse wave transmission time closest to the current pulse wave transmission time, and use calibration data in which the pulse wave transmission time closest to the current pulse wave transmission time exists as optimal calibration data; and the determining unit is configured to use a function as the optimal function, where the function is determined according to the optimal calibration data.

With reference to the second aspect, in a twelfth possible implementation of the second aspect, the second obtaining module includes a first obtaining unit and a second obtaining unit, where the first obtaining unit is configured to obtain an identity of the user, and the second obtaining unit is configured to obtain the second calibration data from multiple pieces of pre-stored calibration data according to the identity of the user that is obtained by the first obtaining unit.

With reference to the twelfth possible implementation of the second aspect, in a thirteenth possible implementation of the second aspect, the first obtaining unit is configured to determine the identity of the user according to at least one of a first electrocardiosignal or a first pulse wave signal in the first calibration data of the user, or the first obtaining unit is configured to determine the identity of the user according to at least one of a current electrocardiosignal or a current pulse wave signal generated when the user currently uses the cuff-less blood pressure measurement apparatus.

With reference to the second aspect, in a fourteenth possible implementation of the second aspect, the calculation module includes a first calculation unit and a second calculation unit, where the first calculation unit is configured to obtain a current electrocardiosignal and a current pulse wave signal generated when the user currently performs measurement by using the cuff-less blood pressure measurement apparatus, and calculate the current pulse wave transmission time; and the second calculation unit is configured to calculate the current blood pressure value of the user according to the optimal function and the current pulse wave transmission time.

According to a third aspect, a cuff-less blood pressure measurement apparatus is provided, and the cuff-less blood pressure measurement apparatus includes a processor, a memory, and a receiver, and the processor is coupled to both the memory and the receiver, where the processor is configured to control the receiver to receive first calibration data of a user, where the first calibration data is data generated when the user performs a manual calibration process before measuring blood pressure by using the cuff-less blood pressure measurement apparatus; the processor is configured to obtain pre-stored second calibration data of the user; determine, according to the first calibration data and the second calibration data, an optimal function used to represent a relationship between a pulse wave transmission time and a blood pressure value of the user; obtain a current pulse wave transmission time of the user; and calculate a current blood pressure value of the user according to the current pulse wave transmission time and the optimal function; and the memory is configured to store the first calibration data and the second calibration data.

With reference to the third aspect, in a first possible implementation of the third aspect, the processor is configured to determine a first function according to the first calibration data, determine a second function according to the second calibration data, determine a degree of difference between the first function and the second function, and determine the optimal function according to the degree of difference.

With reference to the first possible implementation of the third aspect, in a second possible implementation of the third aspect, the processor is configured to determine the first function according to the first calibration data by using a least square method, and determine the second function according to the second calibration data by using the least square method.

With reference to the first possible implementation of the third aspect, in a third possible implementation of the third aspect, the processor is configured to use a third function as the optimal function when the degree of difference is less than a first preset threshold, where the third function is determined by using the first calibration data and the second calibration data.

With reference to the third possible implementation of the third aspect, in a fourth possible implementation of the third aspect, the processor is configured to use the second function as the optimal function when the degree of difference is greater than the first preset threshold and a quantity of samples of the first calibration data is less than a second preset threshold, or the processor is configured to use the first function as the optimal function when the degree of difference is greater than the first preset threshold and a quantity of samples of the first calibration data is greater than the second preset threshold.

With reference to the third possible implementation of the third aspect, in a fifth possible implementation of the third aspect, the processor is configured to use the third function as the optimal function when the degree of difference is greater than the first preset threshold and a quantity of samples of the first calibration data is less than a second preset threshold, where the third function is determined by using the first calibration data and the second calibration data; or the processor is configured to remove an abnormal data point from the first calibration data, and use a fourth function as the optimal function when the degree of difference is greater than the first preset threshold and a quantity of samples of the first calibration data is greater than the second preset threshold, where the fourth function is calculated by using a combination of the second calibration data and remaining first calibration data.

With reference to the third aspect, in a sixth possible implementation of the third aspect, the processor is configured to determine a first function according to the first calibration data, determine a third function according to a combination of the first calibration data and the second calibration data, determine a degree of difference between the first function and the third function, and determine the optimal function according to the degree of difference.

With reference to the sixth possible implementation of the third aspect, in a seventh possible implementation of the third aspect, the processor is configured to determine the first function according to the first calibration data by using a least square method, and determine a second function according to the second calibration data by using the least square method.

With reference to the sixth possible implementation of the third aspect, in an eighth possible implementation of the third aspect, the processor is configured to use the third function as the optimal function when the degree of difference is less than a first preset threshold.

With reference to the eighth possible implementation of the third aspect, in a ninth possible implementation of the third aspect, the processor is configured to use a second function as the optimal function when the degree of difference is greater than the first preset threshold and a quantity of samples of the first calibration data is less than a second preset threshold, where the second function is determined by using the second calibration data; or the processor is configured to use the first function as the optimal function when the degree of difference is greater than the first preset threshold and a quantity of samples of the first calibration data is greater than the second preset threshold.

With reference to the eighth possible implementation of the third aspect, in a tenth possible implementation of the third aspect, the processor is configured to use the third function as the optimal function when the degree of difference is greater than the first preset threshold and a quantity of samples of the first calibration data is less than a second preset threshold; or the processor is configured to remove an abnormal data point from the first calibration data and use a fourth function as the optimal function when the degree of difference is greater than the first preset threshold and a quantity of samples of the first calibration data is greater than the second preset threshold, where the fourth function is calculated by using a combination of the second calibration data and remaining first calibration data.

With reference to the third aspect, in an eleventh possible implementation of the third aspect, the first calibration data and the second calibration data separately include at least one group of a blood pressure value and a corresponding pulse wave transmission time; and the processor is configured to obtain a current pulse wave transmission time of the user; select, from the first calibration data and the second calibration data, a pulse wave transmission time closest to the current pulse wave transmission time, and use calibration data in which the pulse wave transmission time closest to the current pulse wave transmission time exists as optimal calibration data; and use a function as the optimal function, where the function is determined according to the optimal calibration data.

With reference to the third aspect, in a twelfth possible implementation of the third aspect, the processor is configured to obtain an identity of the user, and obtain the second calibration data from multiple pieces of pre-stored calibration data according to the identity of the user.

With reference to the twelfth possible implementation of the third aspect, in a thirteenth possible implementation of the third aspect, the processor is configured to determine the identity of the user according to at least one of a first electrocardiosignal or a first pulse wave signal in the first calibration data of the user, or the processor is configured to determine the identity of the user according to at least one of a current electrocardiosignal or a current pulse wave signal generated when the user currently uses the cuff-less blood pressure measurement apparatus.

With reference to the third aspect, in a fourteenth possible implementation of the third aspect, the processor is configured to obtain a current electrocardiosignal and a current pulse wave signal generated when the user currently performs measurement by using the cuff-less blood pressure measurement apparatus; calculate the current pulse wave transmission time; and calculate the current blood pressure value of the user according to the optimal function and the current pulse wave transmission time.

According to the technical solutions, first calibration data generated when a user performs manual calibration and pre-stored second calibration data are combined, and an optimal function used to represent a function relationship between a pulse wave transmission time and a blood pressure value of the user is determined according to the first calibration data and the second calibration data. In such a manner, when different users wear a cuff-less blood pressure measurement apparatus for measurement, automatic calibration can be performed with reference to pre-stored calibration data to determine an optimal function, so that the pre-stored calibration data can be fully used, and calibration is more accurate. Therefore, a measurement result of a blood pressure value is more accurate.

### BRIEF DESCRIPTION OF DRAWINGS

FIG. 1 is a flowchart of a blood pressure measurement data processing method according to an embodiment of the present disclosure;

FIG. 2 is a schematic diagram of an electrocardiosignal and a pulse wave signal according to an embodiment of the present disclosure;

FIG. 3 is a flowchart of an implementation of determining an optimal function used to represent a relationship between a pulse wave transmission time and a blood pressure value of a user according to an embodiment of the present disclosure;

FIG. 4 is a flowchart of another implementation of determining an optimal function used to represent a relationship between a pulse wave transmission time and a blood pressure value of a user according to an embodiment of the present disclosure;

FIG. 5 is a flowchart of still another implementation of determining an optimal function used to represent a relationship between a pulse wave transmission time and a blood pressure value of a user according to an embodiment of the present disclosure;

FIG. 6 is a flowchart of a manner in which a cuff-less blood pressure measurement apparatus obtains second calibration data pre-stored by a user according to an embodiment of the present disclosure;

FIG. 7 is a flowchart of a manner of determining an identity of a user by using at least one of a first electrocardiosignal or a first pulse wave signal according to an embodiment of the present disclosure;

FIG. 8 is a schematic diagram of a first type of calibration data fitting result according to an embodiment of the present disclosure;

FIG. 9 is a schematic diagram of a second type of calibration data fitting result according to an embodiment of the present disclosure;

FIG. 10 is a schematic diagram of a third type of calibration data fitting result according to an embodiment of the present disclosure;

FIG. 11 is a schematic diagram of a fourth type of calibration data fitting result according to an embodiment of the present disclosure;

FIG. 12 is a schematic structural diagram of a cuff-less blood pressure measurement apparatus according to an embodiment of the present disclosure;

FIG. 13 is a schematic structural diagram of a second obtaining module of a cuff-less blood pressure measurement apparatus according to an embodiment of the present disclosure;

FIG. 14 is a schematic structural diagram of a determining module of a cuff-less blood pressure measurement apparatus according to an embodiment of the present disclosure;

FIG. 15 is another schematic structural diagram of a determining module of a cuff-less blood pressure measurement apparatus according to an embodiment of the present disclosure;

FIG. 16 is still another schematic structural diagram of a determining module of a cuff-less blood pressure measurement apparatus according to an embodiment of the present disclosure;

FIG. 17 is a schematic structural diagram of a calculation module of a cuff-less blood pressure measurement apparatus according to an embodiment of the present disclosure; and

FIG. 18 is a schematic structural diagram of another cuff-less blood pressure measurement apparatus according to an embodiment of the present disclosure.

### DESCRIPTION OF EMBODIMENTS

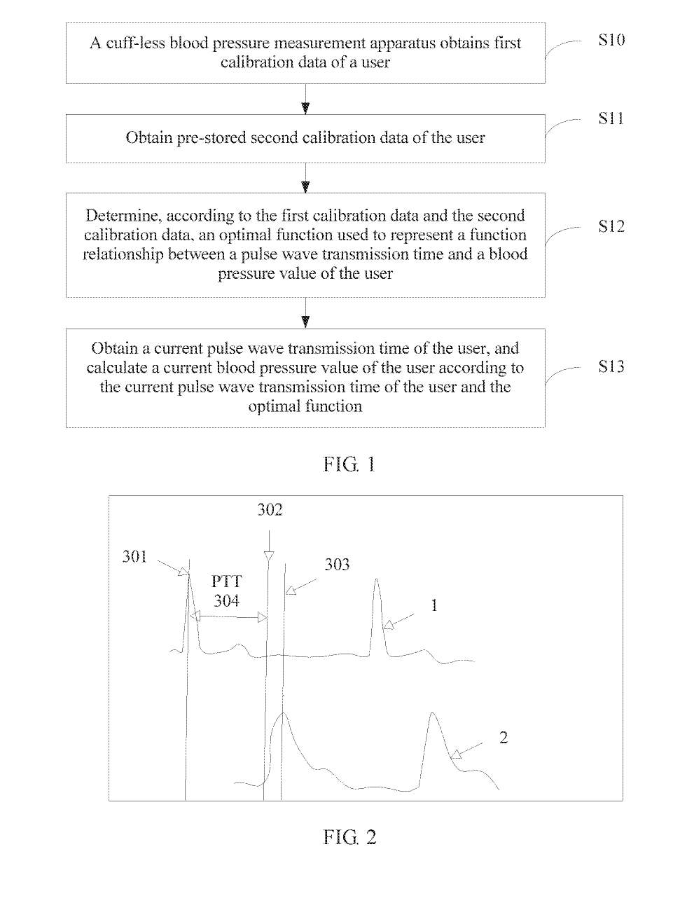

Referring to FIG. 1, FIG. 1 is a flowchart of a blood pressure measurement data processing method according to an embodiment of the present disclosure. As shown in the figure, the blood pressure measurement data processing method in this embodiment includes the following steps.

S10: A cuff-less blood pressure measurement apparatus obtains first calibration data of a user.

The first calibration data is data generated when the user performs a manual calibration process before measuring blood pressure by using the cuff-less blood pressure measurement apparatus. The first calibration data includes at least a first blood pressure value and a first pulse wave transmission time.

The manual calibration process is as follows. The user obtains the first blood pressure value by performing measurement by using a cuff sphygmomanometer. A first electrocardiosignal of the user is collected by using an electrocardiography sensor of the cuff-less blood pressure measurement apparatus, and a first pulse wave signal of the user is collected by using at least one of a light sensor, a pressure sensor, a sound sensor, a photoelectric sensor, an acceleration sensor, or a displacement sensor of the cuff-less blood pressure measurement apparatus. The first pulse wave transmission time is calculated according to the first electrocardiosignal and the first pulse wave signal of the user. Each time the user performs manual calibration, a group of a first blood pressure value and a first pulse wave transmission time is generated. The cuff-less blood pressure measurement apparatus receives the first blood pressure value and the first pulse wave transmission time that are manually input by the user or obtains the first blood pressure value and the first pulse wave transmission time from a cuff blood pressure measurement apparatus by using a specific interface such as Bluetooth or an infrared interface, so as to form the first calibration data.

In a possible implementation in which the first pulse wave transmission time is calculated according to the first electrocardiosignal and the first pulse wave signal, the first pulse wave transmission time is calculated according to a time difference between a reference point on the first electrocardiosignal and a reference point on the first pulse wave signal in a same period as the reference point on the first electrocardiosignal.

In a specific example in which a pulse wave transmission time is calculated according to an electrocardiosignal and a pulse wave signal, referring to FIG. 2, FIG. 2 is a schematic diagram of an electrocardiosignal and a pulse wave signal according to this embodiment of the present disclosure. As shown in the figure, the pulse wave signal in this embodiment is a photoplethysmogram 2 collected by using the photoelectric sensor. A reference point is a vertex, a bottom point, or an intermediate point. A vertex of the electrocardiosignal is 301, and a bottom point and a vertex of the photoplethysmogram are 302 and 303. A pulse wave transmission time (PTT) 304 is calculated according to a time difference between the reference 301 on the electrocardiosignal 1 and the reference point 302 on the pulse wave signal in a same period as the reference point 301.

S11: Obtain pre-stored second calibration data of the user.

The second calibration data includes at least a second blood pressure value and a second pulse wave transmission time. The second calibration data may be historical manual calibration data generated before the first calibration data is obtained and when the user performs a manual calibration process by using the cuff-less blood pressure measurement apparatus. The historical manual calibration data is stored in the cuff-less blood pressure measurement apparatus. Alternatively, the second calibration data may be calibration data pre-stored by the user on a cloud side. The calibration data stored on the cloud side may be calibration data exported from an intelligent wearable device such as a wristwatch. The cuff-less blood pressure measurement apparatus obtains, from the cloud side by using an internal interface, the calibration data stored on the cloud side, and uses the calibration data as the second calibration data.

S12: Determine, according to the first calibration data and the second calibration data, an optimal function used to represent a function relationship between a pulse wave transmission time and a blood pressure value of the user.

In this embodiment of the present disclosure, determining an optimal function used to represent a function relationship between a pulse wave transmission time and a blood pressure value of the user means determining a calibration parameter (or a calibration coefficient) for calculating the blood pressure value according to the pulse wave transmission time. For example, systolic blood pressure (SBP) is calculated by using a formula SBP=a1×PTT+b1, and diastolic blood pressure (DBP) is calculated according to a formula DBP=a2×PTT+b2. The determining the optimal function in this embodiment of the present disclosure is determining values of a1, b1, a2, and b2 in the formulas, and the blood pressure value of the user is accordingly calculated according to current measurement data and the determined calibration parameter.

S13: Obtain a current pulse wave transmission time of the user, and calculate a current blood pressure value of the user according to the current pulse wave transmission time of the user and the optimal function.

The determined optimal function is used to represent the function relationship between the pulse wave transmission time and the blood pressure value of the user. Therefore, when the user currently performs measurement by using the cuff-less blood pressure measurement apparatus, a current electrocardiosignal of the user is collected by using the electrocardiography sensor of the cuff-less blood pressure measurement apparatus, and a current pulse wave signal of the user is collected by using at least one of the light sensor, the pressure sensor, the sound sensor, the photoelectric sensor, the acceleration sensor, or the displacement sensor of the cuff-less blood pressure measurement apparatus. The current pulse wave transmission time is calculated according to the current electrocardiosignal and the current pulse wave signal of the user. The current blood pressure value of the user can be calculated with reference to the determined optimal function according to the calculated current pulse wave transmission time of the user.

In the foregoing embodiment of the present disclosure, calculating systolic blood pressure according to a formula SBP=a1×PTT+b1, and calculating diastolic blood pressure according to a formula DBP=a2×PTT+b2 is merely an implementation example of the present disclosure. Blood pressure may be calculated by using other formulas. For example, the formulas include but are not limited to the following formulas:

$$\begin{matrix}BP = {A}_{1} ln ( {PTT}_{R} ) + {B}_{1} & ( 1 ) \\ BP = {A}_{2} ln ( \frac{{C}_{2}}{{PTT}_{R}^{2}} + 1 ) + {B}_{2} & ( 2 ) \\ BP = \frac{{A}_{3}}{{PTT}_{R}^{2}} + {B}_{3} & ( 3 ) \\ BP = {A}_{4} {PTT}_{R} + {B}_{4} & ( 4 ) \\ BP = {A}_{5} \frac{1}{{PTT}_{R}} + {B}_{5} & ( 5 ) \\ BP = {A}_{6} ( \frac{RT}{{PTT}_{R}^{2}} ) + {B}_{6} . & ( 6 )\end{matrix}$$

In the foregoing formulas, A2=μ×ln(PTT_{w0})
B2=−(SBP₀−DBP₀)×PTT_{w0}/3
C2=SBP₀/3+2DBP₀/3
D2=(SBP₀−DBP₀)×PTT_{w0}²

In the formulas, SBP is systolic blood pressure, DBP is diastolic blood pressure, μ is a vascular characteristic parameter and usually taken as a constant, and a subscript o indicates a calibration value.

According to the blood pressure measurement data processing method provided in the foregoing embodiment of the present disclosure, first calibration data generated when a user performs manual calibration and pre-stored second calibration data are combined, and an optimal function used to represent a relationship between a pulse wave transmission time and a blood pressure value of the user is determined according to the first calibration data and the second calibration data. In such a manner, when a user wears a cuff-less blood pressure measurement apparatus for measurement, automatic calibration can be performed with reference to pre-stored calibration data to determine an optimal function, so that the pre-stored calibration data can be fully used, and calibration is more accurate. Therefore, a measurement result of a blood pressure value of the user is more accurate.

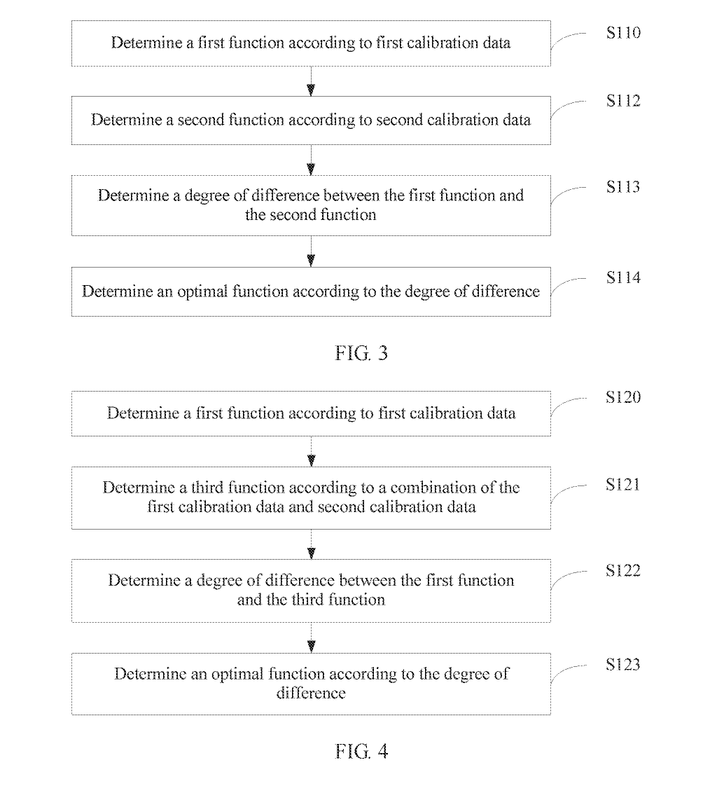

In the foregoing embodiment of the present disclosure, there are three possible implementations of determining the optimal function used to represent the relationship between the pulse wave transmission time and the blood pressure value of the user according to the first calibration data and the second calibration data. The following further describes the three possible implementations with reference to FIG. 3, FIG. 4, and FIG. 5.

For a first possible implementation, refer to FIG. 3. FIG. 3 is a flowchart of an implementation of determining the optimal function used to represent the relationship between the pulse wave transmission time and the blood pressure value of the user according to this embodiment of the present disclosure. As shown in the figure, the following substeps are included.

S110: Determine a first function according to the first calibration data.

The first function herein is a function used to represent a relationship between a first blood pressure value and a first pulse wave transmission time in the first calibration data.

S112: Determine a second function according to the second calibration data.

The second function herein is a function used to represent a relationship between a second blood pressure value and a second pulse wave transmission time in the second calibration data.

In a possible implementation solution, the first function is determined according to the first calibration data by using a least square method. The second function is determined according to the second calibration data by using the least square method.

S113: Determine a degree of difference between the first function and the second function.

The degree of difference between the first function and the second function may be measured by using a linear relationship for associating the two functions in a same coordinate system. After the first function and the second function are determined by using the least square method, a slope change rate and/or a fitting coefficient change rate of the first function relative to the second function are/is determined to determine the degree of difference.

S114: Determine the optimal function according to the degree of difference.

After the degree of difference between the two functions is determined, the optimal function used to represent the relationship between the pulse wave transmission time and the blood pressure value of the user is determined according to the degree of difference.

In an implementation, if the degree of difference is less than a first preset threshold, a third function determined by using the first calibration data and the second calibration data is used as the optimal function. The second function is used as the optimal function if the degree of difference is greater than the first preset threshold and a quantity of samples of the first calibration data is less than a second preset threshold. Alternatively, the first function is used as the optimal function if the degree of difference is greater than the first preset threshold and a quantity of samples of the first calibration data is greater than a second preset threshold. The first preset threshold and the second preset threshold herein may be thresholds that are preset and stored by the user in the cuff-less blood pressure measurement apparatus. The user may adjust the first preset threshold and the second preset threshold according to a requirement. When the degree of difference is indicated by using the slope change rate and the fitting coefficient change rate of the first function relative to the second function, the first preset threshold correspondingly includes two thresholds (which are respectively a slope change rate threshold and a fitting coefficient change rate threshold). For example, the slope change rate threshold is 30%, and the fitting system change rate threshold is 10%. In addition, the second preset threshold is for the quantity of samples that may be set to, for example, 4 or 6.

That is, when the degree of difference is less than the first preset threshold, it may be determined that a deviation between the first calibration data and the pre-stored second calibration data is not large, and the function determined by using a combination of the two pieces of calibration data may be used as the optimal function. If the degree of difference is greater than the first preset threshold, it is determined that a deviation between the first calibration data and the pre-stored second calibration data is relatively large. In this case, determining needs to be further performed with reference to the quantity of samples of the first calibration data. If the quantity of samples of the first calibration data is large enough (exceeds the second preset threshold), the first function determined by using the first calibration data may be independently used as the optimal function. If the quantity of samples of the first calibration data is relatively small (does not exceed the second preset threshold), the second function determined by using the pre-stored second calibration data is directly used as the optimal function.

In addition, other implementations may be as follows. If the degree of difference is less than a first preset threshold, a third function determined by using the first calibration data and the second calibration data is used as the optimal function. If the degree of difference is greater than the first preset threshold and a quantity of samples of the first calibration data is less than a second preset threshold, the third function determined by using the first calibration data and the second calibration data is used as the optimal function. Alternatively, if the degree of difference is greater than the first preset threshold and a quantity of samples of the first calibration data is greater than a second preset threshold, an abnormal data point is removed from the first calibration data, and a fourth function calculated by using a combination of the second calibration data and remaining first calibration data is used as the optimal function.

In an implementation, when the degree of difference is less than the first preset threshold, it may be determined that a deviation between the first calibration data and the pre-stored second calibration data is not large, and the function determined by using a combination of the two pieces of calibration data may be used as the optimal function. When the degree of difference is greater than the first preset threshold and the quantity of samples of the first calibration data is less than the second preset threshold, because a quantity of samples of the second calibration data is small, a difference brought by the second calibration data may be ignored, and the third function determined by using the combination of the first calibration data and the second calibration data is used as the optimal function. If the degree of difference is greater than the first preset threshold and the quantity of samples of the first calibration data is greater than the second preset threshold, a deviation of the first calibration data relative to the pre-stored second calibration data is relatively large, and the quantity of samples of the first calibration data is relatively large. In this case, residual analysis may be further performed on the first calibration data to determine whether there is an abnormal point in the first calibration data. If there is an abnormal point, after the abnormal point is removed, the fourth function calculated by using the combination of the second calibration data and the remaining first calibration data is used as the optimal function.

Referring to FIG. 4, FIG. 4 is a flowchart of another implementation of determining the optimal function used to represent the relationship between the pulse wave transmission time and the blood pressure value of the user according to this embodiment of the present disclosure. A main difference between this implementation solution and the embodiment shown in FIG. 3 lies in that when a degree of difference is determined, the degree of difference is a degree of difference of a third function relative to a first function, where the third function is determined by using a combination of the first calibration data and the second calibration data and the first function is determined by using the first calibration data. As shown in the figure, the following substeps are included.

S120: Determine the first function according to the first calibration data.

The first function herein is a function used to represent a relationship between a first blood pressure value and a first pulse wave transmission time in the first calibration data.

S121: Determine the third function according to the combination of the first calibration data and the second calibration data.

The third function herein is a function used to represent a relationship between a blood pressure value and a pulse wave transmission time in the combination of the first calibration data and the second calibration data.

In a possible implementation solution, the first function is determined according to the first calibration data by using a least square method. The third function is determined according to the first calibration data and the second calibration data by using the least square method.

S122: Determine the degree of difference between the first function and the third function.

The degree of difference between the first function and the third function may be measured by using a linear relationship for associating the two functions in a same coordinate system. Specifically, after the first function and the third function are determined by using the least square method, a slope change rate and/or a fitting coefficient change rate of the third function relative to a second function are/is determined to determine the degree of difference.

S123: Determine the optimal function according to the degree of difference.

After the degree of difference between the two functions is determined, the optimal function used to represent the relationship between the pulse wave transmission time and the blood pressure value of the user is determined according to the degree of difference.

In an implementation, if the degree of difference is less than a first preset threshold, the third function determined by using the first calibration data and the second calibration data is used as the optimal function. A second function is used as the optimal function if the degree of difference is greater than the first preset threshold and a quantity of samples of the first calibration data is less than a second preset threshold. Alternatively, the first function is used as the optimal function if the degree of difference is greater than the first preset threshold and a quantity of samples of the first calibration data is greater than a second preset threshold. The first preset threshold and the second preset threshold herein may be thresholds that are preset and stored by the user in the cuff-less blood pressure measurement apparatus. The user may adjust the first preset threshold and the second preset threshold according to a requirement. When the degree of difference is indicated by using the slope change rate and the fitting coefficient change rate of the first function relative to the second function, the first preset threshold correspondingly includes two thresholds (which are respectively a slope change rate threshold and a fitting coefficient change rate threshold). For example, the slope change rate threshold is 30%, and the fitting system change rate threshold is 10%. In addition, the second preset threshold is for the quantity of samples that may be set to, for example, 4 or 6.

That is, when the degree of difference is less than the first preset threshold, it may be determined that a deviation between the first calibration data and the pre-stored second calibration data is not large, and the function determined by using the combination of the two pieces of calibration data may be used as the optimal function. If the degree of difference is greater than the first preset threshold, it is determined that a deviation between the first calibration data and the pre-stored second calibration data is relatively large. In this case, determining needs to be further performed with reference to the quantity of samples of the first calibration data. If the quantity of samples of the first calibration data is large enough (exceeds the second preset threshold), the first function determined by using the first calibration data may be independently used as the optimal function. If the quantity of samples of the first calibration data is relatively small (does not exceed the second preset threshold), the second function determined by using the pre-stored second calibration data is directly used as the optimal function.

In addition, other implementations may be as follows. If the degree of difference is less than a first preset threshold, the third function determined by using the first calibration data and the second calibration data is used as the optimal function. If the degree of difference is greater than the first preset threshold and a quantity of samples of the first calibration data is less than a second preset threshold, the third function determined by using the first calibration data and the second calibration data is used as the optimal function. Alternatively, if the degree of difference is greater than the first preset threshold and a quantity of samples of the first calibration data is greater than a second preset threshold, an abnormal data point is removed from the first calibration data, and a fourth function calculated by using a combination of the second calibration data and remaining first calibration data is used as the optimal function.

In an implementation, when the degree of difference is less than the first preset threshold, it may be determined that a deviation between the first calibration data and the pre-stored second calibration data is not large, and the function determined by using the combination of the two pieces of calibration data may be used as the optimal function. When the degree of difference is greater than the first preset threshold and the quantity of samples of the first calibration data is less than the second preset threshold, because a quantity of samples of the second calibration data is small, a difference brought by the second calibration data may be ignored, and the third function determined by using the combination of the first calibration data and the second calibration data is used as the optimal function. If the degree of difference is greater than the first preset threshold and the quantity of samples of the first calibration data is greater than the second preset threshold, a deviation of the first calibration data relative to the pre-stored second calibration data is relatively large, and the quantity of samples of the first calibration data is relatively large. In this case, residual analysis may be further performed on the first calibration data to determine whether there is an abnormal point in the first calibration data. If there is an abnormal point, after the abnormal point is removed, the fourth function calculated by using the combination of the second calibration data and the remaining first calibration data is used as the optimal function.

Referring to FIG. 5, FIG. 5 is a flowchart of still another implementation of determining the optimal function used to represent the relationship between the pulse wave transmission time and the blood pressure value of the user according to this embodiment of the present disclosure. As shown in the figure, the following substeps are included.

S130: Obtain a current pulse wave transmission time of the user.

The current pulse wave transmission time of the user may be obtained in the following manner. A corresponding electrocardiosignal and a corresponding pulse wave signal generated when the user currently uses the cuff-less blood pressure measurement apparatus are obtained, and the current pulse wave transmission time of the user is calculated. A current electrocardiosignal of the user is collected by using the electrocardiography sensor of the cuff-less blood pressure measurement apparatus, and a current pulse wave signal of the user is collected by using at least one of the light sensor, the pressure sensor, the sound sensor, the photoelectric sensor, the acceleration sensor, or the displacement sensor of the cuff-less blood pressure measurement apparatus. The current pulse wave transmission time of the user is calculated according to the current electrocardiosignal and the current pulse wave signal of the user.

S131: Select, from the first calibration data and the second calibration data, a pulse wave transmission time closest to the current pulse wave transmission time, and use calibration data in which the pulse wave transmission time closest to the current pulse wave transmission time exists as optimal calibration data.

The obtained current pulse wave transmission time of the user is referred to as a third pulse wave transmission time (PTT3). The current pulse wave transmission time PTT3 is separately compared with a first pulse wave transmission time (PTT1) in the first calibration data and a second pulse wave transmission time (PTT2) in the second calibration data, to determine whether a difference value between the PTT1 and the PTT3 or a difference value between the PTT2 and the PTT3 is smaller. If the difference value between the PTT1 and the PTT3 is smaller, the optimal function is determined by using the first calibration data. If the difference value between the PTT2 and the PTT3 is smaller, the optimal function is determined by using the second calibration data. That is, in this implementation, with reference to the PPT1 in the first calibration data, the PTT2 in the second calibration data, and the PTT3 calculated by using data obtained by means of current measurement, calibration data corresponding to a PTT closest to the PTT3 is used as the optimal calibration data, and a function determined by using the optimal calibration data is used as the optimal function.

Herein, when comparison is performed, an average value of pulse wave transmission times calculated by using multiple pairs of data in the first calibration data may be calculated and used as the first pulse wave transmission time PTT1, an average value of pulse wave transmission times calculated by using multiple pairs of data in the second calibration data may be calculated and used as the second pulse wave transmission time PTT2, and the PTT1 and the PTT2 are separately compared with the PTT3 to determine the optimal calibration data.

Alternatively, a PTT closest to the PTT3 may be found in multiple first pulse wave transmission times PTT1 in the first calibration data and multiple second pulse wave transmission times PTT2 in the second calibration data, and calibration data in which the PTT closest to the PTT3 exists is used as the optimal calibration data. For example, there are multiple first pulse wave transmission times A, B, C, and D in the first calibration data, and there are multiple second pulse wave transmission times A1, B1, C1, and D1 in the second calibration data. If one of A, B, C, or D is closest to the PTT3, the first calibration data is used as the optimal calibration data. If one of A1, B1, C1, or D1 is closest to the PTT3, the second calibration data is used as the optimal calibration data.

S132: Use a function as the optimal function used to represent the relationship between the pulse wave transmission time and the blood pressure value of the user, where the function is determined according to the optimal calibration data.

After the optimal calibration data is determined, the function determined according to the optimal calibration data by using a least square method is used as the optimal function used to represent the relationship between the pulse wave transmission time and the blood pressure value of the user.

In the foregoing implementation solutions, in the implementation solutions shown in FIG. 3 and FIG. 4, when calibration is performed, only the first calibration data generated when the user performs manual calibration and the pre-stored second calibration data are considered. After the functions are determined by using the first calibration data and the second calibration data, the optimal function is determined according to the degree of difference.

In the implementation solution shown in FIG. 5, when calibration is performed, in addition to considering the first calibration data generated when manual calibration is performed and the pre-stored second calibration data, comprehensive consideration is further performed with reference to current measurement data to determine the optimal function. That is, when a pulse wave transmission time in the first calibration data is a PTT1, a pulse wave transmission time in the second calibration data is a PTT2, and a pulse wave transmission time calculated by using an electrocardiosignal and a pulse wave signal correspondingly obtained when the user currently measures blood pressure by using the cuff-less blood pressure measurement apparatus is a PTT3, if the PTT3 is close to the PTT1, calibration is performed by using the first calibration data to obtain the optimal function and calculate the blood pressure value; or if the PTT is close to the PTT2, calibration is performed by using the second calibration data to obtain the optimal function and calculate the blood pressure value.

In the foregoing technical solutions of the present disclosure, the first calibration data or the second calibration data may not exist, or neither the first calibration data nor the second calibration data exists. When the first calibration data does not exist, calibration is performed by using the second calibration data to determine the optimal function. When the second calibration data does not exist, calibration is performed by using the first calibration data to determine the optimal function. If neither the first calibration data nor the second calibration data exists, in this case, the user is prompted to perform manual calibration.

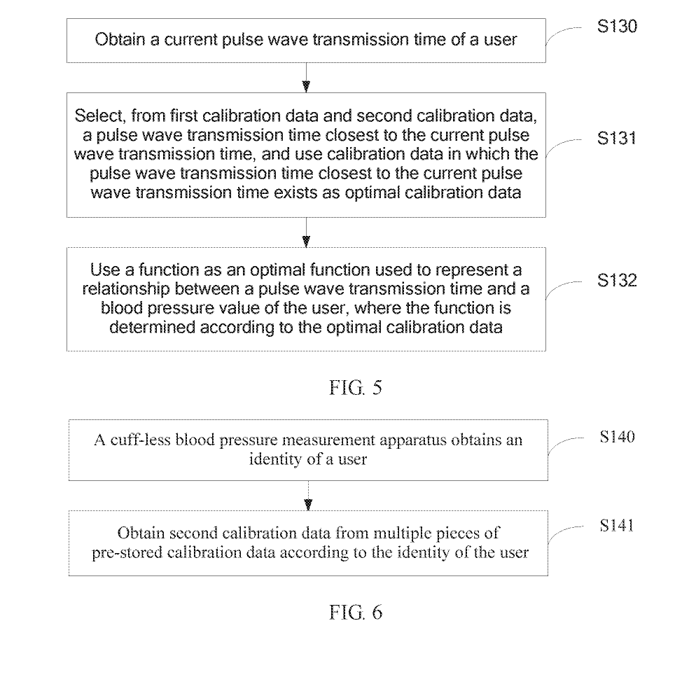

Further referring to FIG. 6, in the foregoing embodiment, a manner in which the cuff-less blood pressure measurement apparatus obtains the second calibration data pre-stored by the user in the cuff-less blood pressure measurement apparatus may include the following substeps.

S140: The cuff-less blood pressure measurement apparatus obtains an identity of the user.

The cuff-less blood pressure measurement apparatus may determine the identity of the user according to one or two of a first electrocardiosignal or a first pulse wave signal in the first calibration data of the user, or determine the identity of the user according to one or two of a current electrocardiosignal or a current pulse wave signal generated when the user currently performs measurement by using the cuff-less blood pressure measurement apparatus.

Although both a feature of an electrocardiosignal and a feature of a pulse wave signal of each individual become different as a detected part and a detection moment change, an electrocardiosignal and a pulse wave signal of a same person basically remain stable. Different individuals have significantly different electrocardiosignals and pulse wave signals. Therefore, the identity of the user may be determined by using the electrocardiosignal or the pulse wave signal.

S141: Obtain the second calibration data from multiple pieces of pre-stored calibration data according to the identity of the user.

After the identity of the user is determined, the second calibration data is obtained from the multiple pieces of pre-stored calibration data according to the identity of the user. The second calibration data may be obtained according to the identity of the user by using a technical solution in the prior art. This is not limited in the present disclosure.

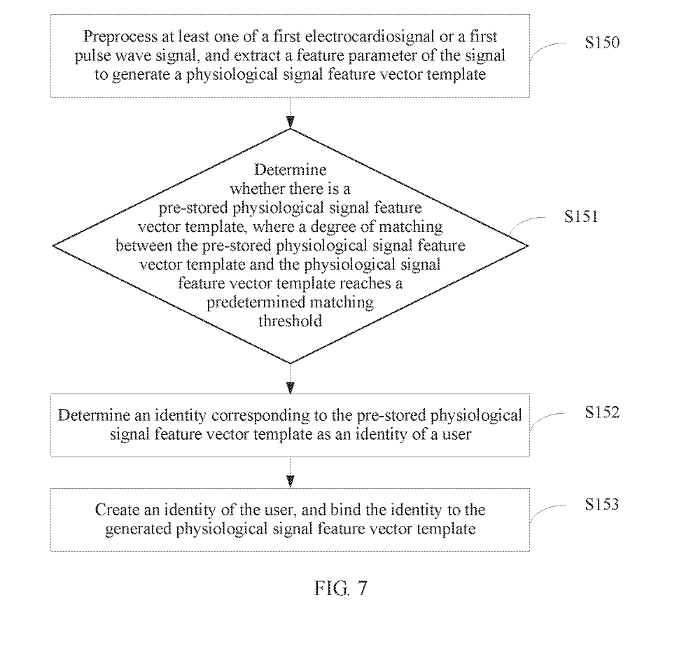

FIG. 7 is a flowchart of a manner of determining the identity of the user by using at least one of a first electrocardiosignal or a first pulse wave signal according to this embodiment of the present disclosure. As shown in the figure, the determining the identity of the user according to at least one of a first electrocardiosignal or a first pulse wave signal includes the following steps.

S150: Preprocess at least one of the first electrocardiosignal or the first pulse wave signal, and extract a feature parameter of the signal to generate a physiological signal feature vector template.

A manner of preprocessing at least one of the first electrocardiosignal or the first pulse wave signal may be but not limited to digital signal conversion, noise reduction, or the like. The extracted feature parameter of the signal may be a vertex, a valley point, or the like of a signal waveform. The physiological signal feature vector template is generated according to the extracted feature parameter.

S151: Determine whether there is a pre-stored physiological signal feature vector template, where a degree of matching between the pre-stored physiological signal feature vector template and the physiological signal feature vector template reaches a preset matching threshold.

The generated physiological signal feature vector template is matched with a pre-stored physiological signal feature vector template, and it is determined whether there is a pre-stored physiological signal feature vector template, where matching between the pre-stored physiological signal feature vector template and the generated physiological signal feature vector template reaches the preset matching threshold. If there is such a pre-stored physiological signal feature vector template, S152 is performed; or if there is no such a pre-stored physiological signal feature vector template, S153 is performed.

The matching threshold herein is a threshold that is used to measure a degree of matching and that is preset and stored by the user in the measurement apparatus, and may be adjusted according to a requirement.

S152: Determine an identity corresponding to the pre-stored physiological signal feature vector template as the identity of the user.

When there is a pre-stored physiological signal feature vector template that matches the generated physiological signal feature vector template, and a degree of matching reaches the preset matching threshold, an identity corresponding to the pre-stored physiological signal feature vector template is determined as the identity of the user.

S153: Create an identity of the user, and bind the identity to the generated physiological signal feature vector template.

When there is no pre-stored physiological signal feature vector template, where a degree of matching between the pre-stored physiological signal feature vector template and the generated physiological signal feature vector module reaches the preset matching threshold, an identity of the user is created, and is bound to the generated physiological signal feature vector template.

In an implementation process, the first calibration data may not exist. For example, the user does not perform a manual calibration process before performing measurement by using the cuff-less blood pressure measurement apparatus. In this case, in a possible implementation, at least one of a corresponding electrocardiosignal or a corresponding pulse wave signal generated when the user currently performs measurement by using the cuff-less blood pressure measurement apparatus may be obtained to determine the identity of the user. An implementation process of determining the identity of the user according to at least one of the electrocardiosignal or the pulse wave signal is similar to a process shown in FIG. 7. Details are not described herein again in the present disclosure.

To further describe the method in the present disclosure, the following provides description by using an example in which a degree of difference between the first calibration data and the second calibration data is determined by using the least square method. In this embodiment of the present disclosure, the systolic blood pressure is calculated according to the formula SBP=a1×PTT+b1, and the diastolic blood pressure is calculated according to the formula DBP=a2×PTT+b2. In the following, a process of determining calibration data a2 and b2 of the diastolic blood pressure is used as an example.

Calibration policy 1: Calibration parameters are estimated with reference to the first calibration data and the second calibration data by using the least square method, and the optimal function is determined according to a result obtained by performing fitting according to the least square method.

When a comparison result obtained by comparing a fitting result of the first calibration data with that of the second calibration data meets the following conditions, it is determined that the calibration data has no significant change, and a first policy is used as a blood pressure calculation policy:

(1) a slope change rate of a trend line is less than a specified threshold v1=30%; and

(2) a change rate of R2 is less than a specified threshold v2=10%, and an updated R2 is greater than a specified threshold v3=0.9.

The first policy is as follows. With reference to the first calibration data and the second calibration data, a result, that is, the third function, obtained by performing linear fitting according to the combination of the first calibration data and the second calibration data by using the least square method is used as representation parameters of the optimal function, the representation parameters are updated, and the blood pressure value of the user is calculated according to the updated representation parameters.

After the representation parameters are updated, the original second calibration data is replaced with updated calibration data (that is, the original stored second calibration data is replaced with the combination of the first calibration data and the second calibration data), and representation parameters, that is, the second function, obtained by using the original second calibration data are replaced with the updated representation parameters.

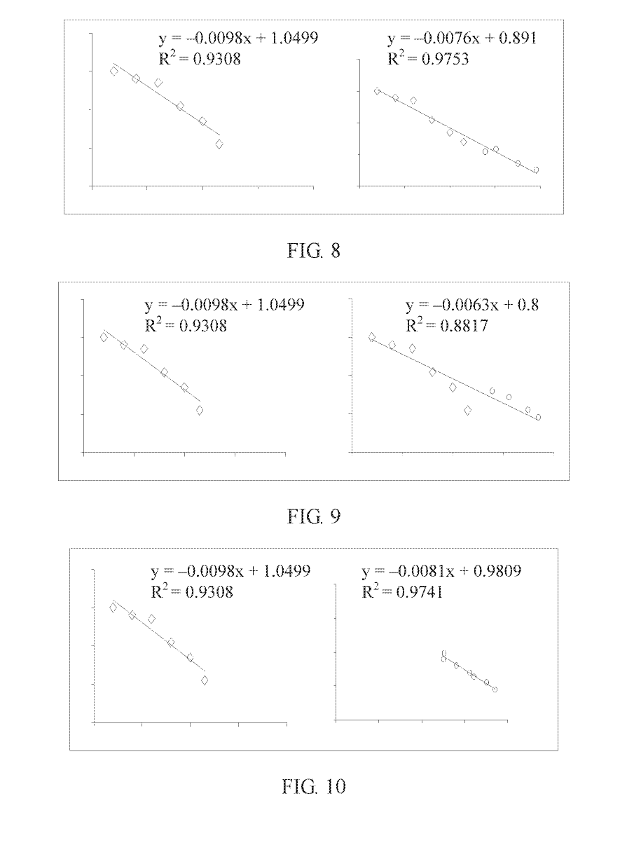

For example, as shown in FIG. 8, in FIG. 8, a graph on the left is original calibration data, that is, the second calibration data (a horizontal axis is diastolic blood pressure measured by the cuff sphygmomanometer, and a unit is mmHg; a vertical axis is a PTT value measured by the cuff-less blood pressure measurement apparatus, and a unit is s). A linear fitting result is y=−0.0098x+1.0499, and R²=0.9308. A circle point in a graph on the right is updated calibration data, that is, the first calibration data. (In this embodiment, the user performs recalibration four times by using the cuff sphygmomanometer. The graph on the left is the original calibration data. Each circle point in the graph on the right represents one calibration. Each calibration process is as follows. The user measures the diastolic blood pressure and the systolic blood pressure by using the cuff sphygmomanometer, and after resting 30 s, wears the cuff-less blood pressure measurement apparatus to measure a PTT). With reference to the existing calibration data and the updated calibration data, a result obtained by performing re-fitting is y=−0.0076x+0.891, and R²=0.9753. Recalculated representation parameters have no significant change:

a slope change rate is |−0.0098−(−0.0076)|/|−0.0098|=23.45% and is less than v1=30%; and

a change rate of R² is|0.9308−0.9753)|/0.9308=4.78% and is less than v2=10%, and an updated R²=0.9753 and is greater than v3=0.9.

The representation parameters are updated to a2=−0.0076 and b2=0.8910 (that is, the third function). The blood pressure value of the user is calculated according to the updated representation parameters, the updated calibration data is stored, and the original calibration data is replaced with the updated calibration data.

When a comparison result obtained by comparing a fitting result of the first calibration data with that of the second calibration data meets one of the following conditions, it is determined that the calibration data significantly changes:

(1) a slope change rate of a trend line is greater than v1=30%; and

(2) a change rate of R² is greater than a specified threshold v2=10%, or R²≦0.9.

In this case, a quantity of samples of updated calibration data needs to be further determined. If the quantity of samples of the updated calibration data is less than a specified threshold, a second policy is used as a blood pressure calculation policy.

The second policy is as follows. The representation parameters are not updated, the second function determined by using the original stored calibration data, that is, the second calibration data, is used as the optimal function to calculate blood pressure, and calibration data updated this time, that is, the first calibration data, is not stored. In this case, a prompt that abnormal calibration data may exist may be sent to the user.

As shown in FIG. 9, in FIG. 9, the quantity of samples of the updated calibration data is less than a specified threshold 6 (in this embodiment, the user performs recalibration four times by using the cuff sphygmomanometer). A linear fitting result is y=−0.0098x+1.0499, and R²=0.9308. Each circle point in a graph on the right represents one calibration. (Each calibration process is as follows. The user measures the diastolic blood pressure and the systolic blood pressure by using the cuff sphygmomanometer, and after resting 30 s, wears the cuff-less blood pressure measurement apparatus to measure a PTT). A fitting result obtained by performing fitting with reference to the two groups of calibration data is y=−0.0063x+0.8, and R²=0.8817. The fitting results are compared, and it is determined that the calibration data significantly changes and that the quantity of samples of the updated calibration data is less than the specified threshold. In this case, a corresponding calibration policy is as follows. The calibration data is not updated, the blood pressure value of the user is calculated according to an original stored function (that is, the second function determined by using the second calibration data) a2=−0.0098 and b2=1.0499, and the calibration data updated this time, that is, the first calibration data, is not stored.

If the quantity of samples of the updated calibration data is greater than the specified threshold, a third policy is used as a blood pressure calculation policy.

The third policy is as follows. Calibration parameters (that is, the first function) determined by using the updated calibration data are used as the optimal function, and the original stored calibration data and the representation parameters are replaced with the updated calibration data and the optimal function.

As shown in FIG. 10, in FIG. 10, the quantity of samples of the updated calibration data is greater than a specified threshold 6. (In this embodiment, the user performs recalibration seven times by using the cuff sphygmomanometer. Each circle point in a graph on the right represents one calibration. Each calibration process is as follows. The user measures the diastolic blood pressure and the systolic blood pressure by using the cuff sphygmomanometer, and after resting 30 s, wears the cuff-less blood pressure measurement apparatus to measure a PTT). A fitting result of the updated calibration data is y=−0.0081x+0.9809, and R²=0.9741. A fitting result of the original calibration data is y=−0.0098x+1.0499, and R²=0.9308. The fitting results are compared, and it is determined that the calibration data significantly changes and that the quantity of samples of the updated calibration data is greater than the specified threshold. In this case, a corresponding calibration policy is as follows. Representation parameters are recalculated according to the updated calibration data: a2=−0.0081 and b2=0.9809 (that is, first representation parameters), the blood pressure value of the user is calculated according to the recalculated representation parameters a2=−0.0081 and b2=0.9809, the original calibration data is deleted, the updated calibration data, that is, the first calibration data, is stored, and the original representation parameters are replaced with the updated calibration parameters, that is, the first function.

When the quantity of samples of the updated calibration data is greater than the specified threshold, in a preferred implementation solution, residual analysis may be performed with reference to the existing calibration data (the second calibration data) and the updated calibration data (the first calibration data) to determine whether there is an abnormal point in the second calibration data. If there is an abnormal point, after the abnormal point is removed, the fourth function calculated by using the combination of the first calibration data and the second calibration data from which the abnormal point is removed is used as the optimal function.

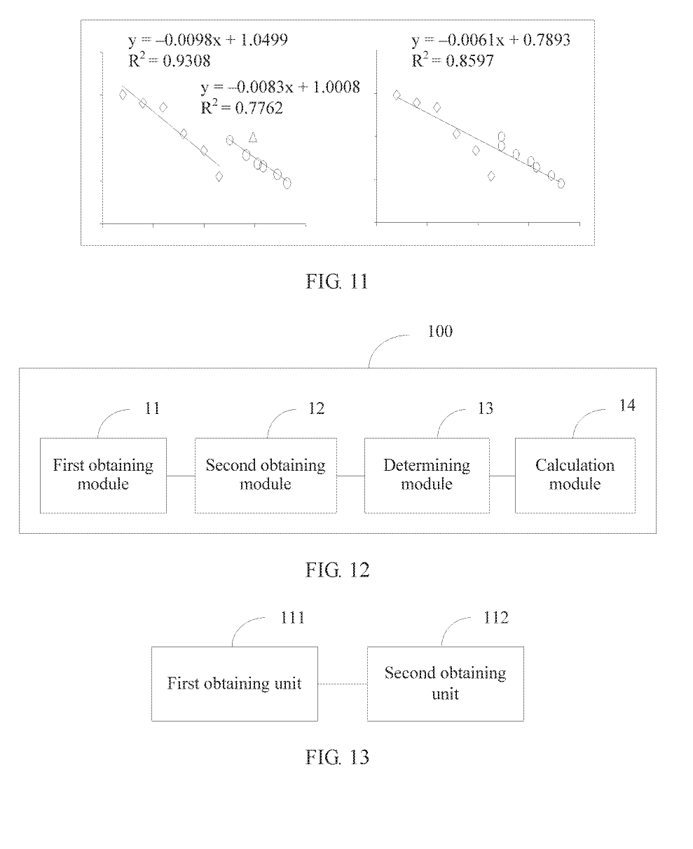

In FIG. 11, a square point in a graph on the left is existing calibration data (a horizontal axis is diastolic blood pressure measured by the cuff sphygmomanometer, and a unit is mmHg; a vertical axis is a PTT value measured by the cuff-less blood pressure measurement apparatus, and a unit is s). A linear fitting result is y=−0.0098x+1.0499, and R²=0.9308. A circle point is updated calibration data (In this embodiment, the cuff-less blood pressure measurement apparatus communicates with a cloud side, and downloads calibration data of the user from the cloud side. The quantity of samples is 8. A linear fitting result of the updated calibration data is y=−0.0083x+1.0008, and R²=0.7762. Residual analysis is performed on the eight groups of calibration data, and one abnormal point—a triangle in the graph is found. The point is deleted, and with reference to the seven remaining groups of calibration data and the six groups of original calibration data, a linear fitting result is y=−0.0061x+0.7893, and R²=0.8597. Representation parameters a2=−0.0061 and b2=0.7893 (the fourth function) are recalculated. The blood pressure value is calculated according to the fourth function.).

The foregoing is detailed description of the blood pressure measurement data processing method provided in this embodiment of the present disclosure. It can be understood that in the present disclosure, an optimal function used to represent a function relationship between a preset biological feature and a blood pressure value of a user is determined according to first calibration data and second calibration data, where the first calibration data is obtained when the user performs a manual calibration process before measuring blood pressure by using a cuff-less blood pressure measurement apparatus, and the second calibration data is pre-stored in the cuff-less blood pressure measurement apparatus. In such a manner, calibration precision can be improved, thereby improving accuracy of a blood pressure measurement result.

In addition, in this embodiment of the present disclosure, an identity of the user may be determined according to a physiological signal (at least one of an electrocardiosignal or a pulse wave signal) of the user, and original stored calibration data of the user may be obtained according to the determined identity of the user. Therefore, a problem in the prior art that calibration data and a calibration parameter can be obtained only with manual selection is resolved, the calibration data and the calibration parameter that are corresponding to the user can be automatically obtained without manual selection, and user experience is improved. In addition, when calibration is performed, the first calibration data generated when the user performs manual calibration and the pre-stored second calibration data are combined, and an optimal function used to represent a function relationship between a pulse wave transmission time and a blood pressure value of the user is determined according to the first calibration data and the second calibration data, so that the pre-stored calibration data can be fully used, and calibration is more accurate. Therefore, a measurement result of the blood pressure value is more accurate.

Further referring to FIG. 12, FIG. 12 is a schematic structural diagram of a cuff-less blood pressure measurement apparatus according to an embodiment of the present disclosure. The cuff-less blood pressure measurement apparatus provided in this embodiment is configured to perform the method described in the foregoing embodiment. As shown in the figure, a cuff-less blood pressure measurement apparatus 100 in this embodiment includes a first obtaining module 11, a second obtaining module 12, a determining module 13, and a calculation module 14.

The first obtaining module 11 is configured to obtain first calibration data, where the first calibration data is data generated when a user performs a manual calibration process before measuring blood pressure by using the cuff-less blood pressure measurement apparatus.

The first calibration data includes at least a first blood pressure value and a first pulse wave transmission time.

The manual calibration process is as follows. The user obtains the first blood pressure value by performing measurement by using a cuff sphygmomanometer. A first electrocardiosignal of the user is collected by using an electrocardiography sensor of the cuff-less blood pressure measurement apparatus, and a first pulse wave signal of the user is collected by using at least one of a light sensor, a pressure sensor, a sound sensor, a photoelectric sensor, an acceleration sensor, or a displacement sensor of the cuff-less blood pressure measurement apparatus. The first pulse wave transmission time is calculated according to the first electrocardiosignal and the first pulse wave signal of the user. Each time the user performs manual calibration, a group of a first blood pressure value and a first pulse wave transmission time is generated. The first obtaining module 11 receives the first blood pressure value and the first pulse wave transmission time that are manually input by the user or obtains the first blood pressure value and the first pulse wave transmission time from a cuff blood pressure measurement apparatus by using a specific interface such as Bluetooth or an infrared interface, so as to form the first calibration data.

In a possible implementation, the first pulse wave transmission time is calculated according to a time difference between a reference point on the first electrocardiosignal and a reference point on the first pulse wave signal in a same period as the reference point on the first electrocardiosignal.

The second obtaining module 12 is configured to obtain pre-stored second calibration data of the user.

The second calibration data includes at least a second blood pressure value and a second pulse wave transmission time. The second calibration data may be historical manual calibration data generated before the first calibration data is obtained and when the user performs a manual calibration process by using the cuff-less blood pressure measurement apparatus. The historical manual calibration data is stored in the cuff-less blood pressure measurement apparatus. Alternatively, the second calibration data may be calibration data pre-stored by the user on a cloud side. The calibration data stored on the cloud side may be calibration data exported from an intelligent wearable device such as a wristwatch. The second obtaining module 12 obtains, from the cloud side by using an internal interface, the calibration data stored on the cloud side, and uses the calibration data as the second calibration data.

Further referring to FIG. 13, FIG. 13 is a schematic structural diagram of the second obtaining module in the cuff-less blood pressure measurement apparatus in this embodiment of the present disclosure. In a possible implementation solution, the second obtaining module includes a first obtaining unit 111 and a second obtaining unit 112.

The first obtaining unit 111 is configured to obtain an identity of the user.

The first obtaining unit 111 may determine the identity of the user according to at least one of a first electrocardiosignal or a first pulse wave signal in the first calibration data, or determine the identity of the user according to at least one of a current electrocardiosignal or a current pulse wave signal generated when the user currently uses the cuff-less blood pressure measurement apparatus.

Although a feature of an electrocardiosignal and a feature of a pulse wave signal of each individual become different as a detected part and a detection moment change, an electrocardiosignal and a pulse wave signal of a same person basically remain stable. Different individuals have significantly different electrocardiosignals and pulse wave signals. Therefore, the identity of the user may be determined by using the electrocardiosignal or the pulse wave signal.

The second obtaining unit 112 is configured to obtain the second calibration data from multiple pieces of pre-stored calibration data according to the identity of the user that is obtained by the first obtaining unit 111.

After the first obtaining unit 111 determines the identity of the user, the second obtaining unit 162 obtains the second calibration data from the multiple pieces of pre-stored calibration data according to the identity of the user.

The determining module 13 is configured to determine, according to the first calibration data and the second calibration data, an optimal function used to represent a relationship between a pulse wave transmission time and a blood pressure value of the user.

In this embodiment of the present disclosure, the determining module 13 determines an optimal function used to represent a function relationship between a pulse wave transmission time and a blood pressure value of the user by determining a calibration parameter (or a calibration coefficient) for calculating the blood pressure value according to the pulse wave transmission time. For example, systolic blood pressure is calculated by using a formula SBP=a1×PTT+b1, and diastolic blood pressure is calculated according to a formula DBP=a2× PTT+b2. Determining the optimal function in this embodiment of the present disclosure corresponds to determining values of a1, b1, a2, and b2 in the formulas, and the blood pressure value of the user is accordingly calculated according to current measurement data and the determined calibration parameter.

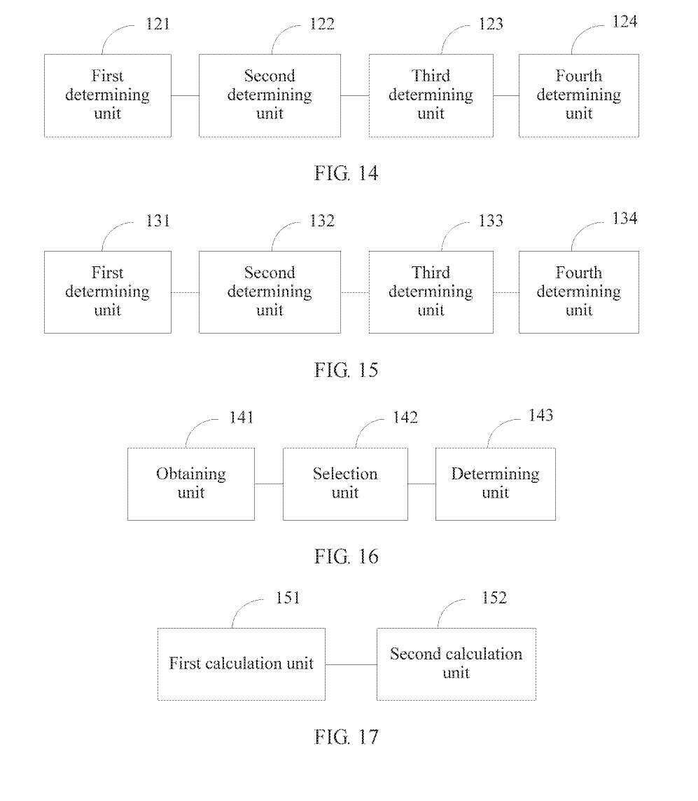

Further referring to FIG. 14, in an embodiment, the determining module in the cuff-less blood pressure measurement apparatus in the foregoing embodiment includes a first determining unit 121, a second determining unit 122, a third determining unit 123, and a fourth determining unit 124.

The first determining unit 121 is configured to determine a first function according to the first calibration data.

The first function herein is a function used to represent a relationship between a first blood pressure value and a first pulse wave transmission time in the first calibration data.

The second determining unit 122 is configured to determine a second function according to the second calibration data.

The second function herein is a function used to represent a relationship between a second blood pressure value and a second pulse wave transmission time in the second calibration data.

In a possible implementation solution, the first determining unit 121 determines the first function according to the first calibration data by using a least square method. The second determining unit 122 determines the second function according to the second calibration data by using the least square method.

The third determining unit 123 is configured to determine a degree of difference between the first function and the second function.

The degree of difference between the first function and the second function may be measured by using a linear relationship for associating the two functions in a same coordinate system. Specifically, after the first function and the second function are determined by using the least square method, a slope change rate and/or a fitting coefficient change rate of the first function relative to the second function are/is determined to determine the degree of difference.

The fourth determining unit 124 is configured to determine the optimal function according to the degree of difference.

After the degree of difference between the two functions is determined, the fourth determining unit 124 determines, according to the degree of difference, the optimal function used to represent the relationship between the pulse wave transmission time and the blood pressure value of the user.

In an implementation, if the degree of difference is less than a first preset threshold, the fourth determining unit 124 uses a third function as the optimal function, where the third function is determined by using the first calibration data and the second calibration data. The fourth determining unit 124 uses the second function as the optimal function if the degree of difference is greater than the first preset threshold and a quantity of samples of the first calibration data is less than a second preset threshold. Alternatively, the fourth determining unit 124 uses the first function as the optimal function if the degree of difference is greater than the first preset threshold and a quantity of samples of the first calibration data is greater than a second preset threshold. The first preset threshold and the second preset threshold herein may be thresholds that are preset and stored by the user in the cuff-less blood pressure measurement apparatus. The user may adjust the first preset threshold and the second preset threshold according to a requirement. When the degree of difference is indicated by using the slope change rate and the fitting coefficient change rate of the first function relative to the second function, the first preset threshold correspondingly includes two thresholds (which are respectively a slope change rate threshold and a fitting coefficient change rate threshold). For example, the slope change rate threshold is 30%, and the fitting system change rate threshold is 10%. In addition, the second preset threshold is for the quantity of samples that may be set to, for example, 4 or 6.

That is, when the degree of difference is less than the first preset threshold, it may be determined that a deviation between the first calibration data and the pre-stored second calibration data is not large, and the function determined by using a combination of the two pieces of calibration data may be used as the optimal function. If the degree of difference is greater than the first preset threshold, it is determined that a deviation between the first calibration data and the pre-stored second calibration data is relatively large. In this case, determining needs to be further performed with reference to the quantity of samples of the first calibration data. If the quantity of samples of the first calibration data is large enough (exceeds the second preset threshold), the first function determined by using the first calibration data may be independently used as the optimal function. If the quantity of samples of the first calibration data is relatively small (does not exceed the second preset threshold), the second function determined by using the pre-stored second calibration data is directly used as the optimal function.

In addition, other implementations may be as follows. If the degree of difference is less than a first preset threshold, the fourth determining unit 124 uses a third function as the optimal function, where the third function is determined by using the first calibration data and the second calibration data. If the degree of difference is greater than the first preset threshold and a quantity of samples of the first calibration data is less than a second preset threshold, the fourth determining unit 124 uses the third function as the optimal function, where the third function is determined by using the first calibration data and the second calibration data. Alternatively, if the degree of difference is greater than the first preset threshold and a quantity of samples of the first calibration data is greater than a second preset threshold, the fourth determining unit 124 removes an abnormal data point from the first calibration data, and uses a fourth function as the optimal function, where the fourth function is calculated by using a combination of the second calibration data and remaining first calibration data.

In an implementation, when the degree of difference is less than the first preset threshold, it may be determined that a deviation between the first calibration data and the pre-stored second calibration data is not large, and the function determined by using a combination of the two pieces of calibration data may be used as the optimal function. When the degree of difference is greater than the first preset threshold and the quantity of samples of the first calibration data is less than the second preset threshold, because a quantity of samples of the second calibration data is small, a difference brought by the second calibration data may be ignored, and the third function determined by using the combination of the first calibration data and the second calibration data is used as the optimal function. If the degree of difference is greater than the first preset threshold and the quantity of samples of the first calibration data is greater than the second preset threshold, a deviation of the first calibration data relative to the pre-stored second calibration data is relatively large, and the quantity of samples of the first calibration data is relatively large. In this case, residual analysis may be further performed on the first calibration data to determine whether there is an abnormal point in the first calibration data. If there is an abnormal point, after the abnormal point is removed, the fourth function calculated by using the combination of the second calibration data and the remaining first calibration data is used as the optimal function.

Further referring to FIG. 15, in another embodiment, the determining module in the cuff-less blood pressure measurement apparatus in the foregoing embodiment includes a first determining unit 131, a second determining unit 132, a third determining unit 133, and a fourth determining unit 134.

The first determining unit 131 is configured to determine a first function according to the first calibration data.

The first function herein is a function used to represent a relationship between a first blood pressure value and a first pulse wave transmission time in the first calibration data.

The second determining unit 132 is configured to determine a third function according to a combination of the first calibration data and the second calibration data.

The third function herein is a function used to represent a relationship between a blood pressure value and a pulse wave transmission time in the combination of the first calibration data and the second calibration data.

In a possible implementation solution, the first determining unit 131 determines the first function according to the first calibration data by using a least square method. The second determining unit 132 determines the third function according to the first calibration data and the second calibration data by using the least square method.

The third determining unit 133 is configured to determine a degree of difference between the first function and the third function.

The degree of difference between the first function and the third function may be measured by using a linear relationship for associating the two functions in a same coordinate system. Specifically, after the first function and the third function are determined by using the least square method, a slope change rate and/or a fitting coefficient change rate of the third function relative to a second function are/is determined to determine the degree of difference.

The fourth determining unit 134 is configured to determine the optimal function according to the degree of difference.

After the degree of difference between the two functions is determined, the fourth determining unit 134 determines, according to the degree of difference, the optimal function used to represent the relationship between the pulse wave transmission time and the blood pressure value of the user.

In an implementation, if the degree of difference is less than a first preset threshold, the fourth determining unit 134 uses the third function as the optimal function, where the third function is determined by using the first calibration data and the second calibration data. The fourth determining unit 134 uses a second function as the optimal function if the degree of difference is greater than the first preset threshold and a quantity of samples of the first calibration data is less than a second preset threshold. Alternatively, the fourth determining unit 134 uses the first function as the optimal function if the degree of difference is greater than the first preset threshold and a quantity of samples of the first calibration data is greater than a second preset threshold. The first preset threshold and the second preset threshold herein may be thresholds that are preset and stored by the user in the cuff-less blood pressure measurement apparatus. The user may adjust the first preset threshold and the second preset threshold according to a requirement. When the degree of difference is indicated by using the slope change rate and the fitting coefficient change rate of the first function relative to the second function, the first preset threshold correspondingly includes two thresholds (which are respectively a slope change rate threshold and a fitting coefficient change rate threshold). For example, the slope change rate threshold is 30%, and the fitting system change rate threshold is 10%. In addition, the second preset threshold is for the quantity of samples that may be set to, for example, 4 or 6.

That is, when the degree of difference is less than the first preset threshold, it may be determined that a deviation between the first calibration data and the pre-stored second calibration data is not large, and the function determined by using the combination of the two pieces of calibration data may be used as the optimal function. If the degree of difference is greater than the first preset threshold, it is determined that a deviation between the first calibration data and the pre-stored second calibration data is relatively large. In this case, determining needs to be further performed with reference to the quantity of samples of the first calibration data. If the quantity of samples of the first calibration data is large enough (exceeds the second preset threshold), the first function determined by using the first calibration data may be independently used as the optimal function. If the quantity of samples of the first calibration data is relatively small (does not exceed the second preset threshold), the second function determined by using the pre-stored second calibration data is directly used as the optimal function.

In addition, other implementations may be as follows If the degree of difference is less than a first preset threshold, the fourth determining unit 134 uses the third function as the optimal function, where the third function is determined by using the first calibration data and the second calibration data. If the degree of difference is greater than the first preset threshold and a quantity of samples of the first calibration data is less than a second preset threshold, the fourth determining unit 134 uses the third function as the optimal function, where the third function is determined by using the first calibration data and the second calibration data. Alternatively, if the degree of difference is greater than the first preset threshold and a quantity of samples of the first calibration data is greater than a second preset threshold, the fourth determining unit 134 removes an abnormal data point from the first calibration data, and uses a fourth function as the optimal function, where the fourth function is calculated by using a combination of the second calibration data and remaining first calibration data.

In an implementation, when the degree of difference is less than the first preset threshold, it may be determined that a deviation between the first calibration data and the pre-stored second calibration data is not large, and the function determined by using the combination of the two pieces of calibration data may be used as the optimal function. When the degree of difference is greater than the first preset threshold and the quantity of samples of the first calibration data is less than the second preset threshold, because a quantity of samples of the second calibration data is small, a difference brought by the second calibration data may be ignored, and the third function determined by using the combination of the first calibration data and the second calibration data is used as the optimal function. If the degree of difference is greater than the first preset threshold and the quantity of samples of the first calibration data is greater than the second preset threshold, a deviation of the first calibration data relative to the pre-stored second calibration data is relatively large, and the quantity of samples of the first calibration data is relatively large. In this case, residual analysis may be further performed on the first calibration data to determine whether there is an abnormal point in the first calibration data. If there is an abnormal point, after the abnormal point is removed, the fourth function calculated by using the combination of the second calibration data and the remaining first calibration data is used as the optimal function.

Further referring to FIG. 16, in still another embodiment, the determining module in the cuff-less blood pressure measurement apparatus in the foregoing embodiment includes an obtaining unit 141, a selection unit 142, and a determining unit 143.

The obtaining unit 141 is configured to obtain a current pulse wave transmission time of the user.

The current pulse wave transmission time of the user may be obtained in the following manner. A corresponding electrocardiosignal and a corresponding pulse wave signal generated when the user currently uses the cuff-less blood pressure measurement apparatus are obtained, and the current pulse wave transmission time of the user is calculated. A current electrocardiosignal of the user is collected by using the electrocardiography sensor of the cuff-less blood pressure measurement apparatus, and a current pulse wave signal of the user is collected by using at least one of the light sensor, the pressure sensor, the sound sensor, the photoelectric sensor, the acceleration sensor, or the displacement sensor of the cuff-less blood pressure measurement apparatus. The current pulse wave transmission time of the user is calculated according to the current electrocardiosignal and the current pulse wave signal of the user.

The selection unit 142 is configured to select, from the first calibration data and the second calibration data, a pulse wave transmission time closest to the current pulse wave transmission time, and use calibration data in which the pulse wave transmission time closest to the current pulse wave transmission time exists as optimal calibration data.

The obtained current pulse wave transmission time of the user is referred to as PTT3. The current pulse wave transmission time PTT3 is separately compared with a first pulse wave transmission time PTT1 in the first calibration data and a second pulse wave transmission time PTT2 in the second calibration data, to determine whether a difference value between the PTT1 and the PTT3 or a difference value between the PTT2 and the PTT3 is smaller. If the difference value between the PTT1 and the PTT3 is smaller, the optimal function is determined by using the first calibration data. If the difference value between the PTT2 and the PTT3 is smaller, the optimal function is determined by using the second calibration data. That is, in this implementation, with reference to the PPT1 in the first calibration data, the PTT2 in the second calibration data, and the PTT3 calculated by using data obtained by means of current measurement, calibration data corresponding to a PTT closest to the PTT3 is used as the optimal calibration data, and a function determined by using the optimal calibration data is used as the optimal function.

Herein, when a comparison is performed, an average value of pulse wave transmission times calculated by using multiple pairs of data in the first calibration data may be calculated and used as the first pulse wave transmission time PTT1, an average value of pulse wave transmission times calculated by using multiple pairs of data in the second calibration data may be calculated and used as the second pulse wave transmission time PTT2, and the PTT1 and the PTT2 are separately compared with the PTT3 to determine the optimal calibration data.

Alternatively, a PTT closest to the PTT3 may be found in multiple first pulse wave transmission times PTT1 in the first calibration data and multiple second pulse wave transmission times PTT2 in the second calibration data, and calibration data in which the PTT closest to the PTT3 exists is used as the optimal calibration data. For example, there are multiple first pulse wave transmission times A, B, C, and D in the first calibration data, and there are multiple second pulse wave transmission times A1, B1, C1, and D1 in the second calibration data. If one of A, B, C, or D is closest to the PTT3, the first calibration data is used as the optimal calibration data. If one of A1, B1, C1, or D1 is closest to the PTT3, the second calibration data is used as the optimal calibration data.

The determining unit 143 is configured to use a function as the optimal function, where the function is determined according to the optimal calibration data.

After the optimal calibration data is determined, the function determined according to the optimal calibration data by using a least square method is used as the optimal function used to represent the relationship between the pulse wave transmission time and the blood pressure value of the user.

In the foregoing technical solutions of the present disclosure, the first calibration data or the second calibration data may not exist, or neither the first calibration data nor the second calibration data exists. When the first calibration data does not exist, the determining module performs calibration by using the second calibration data to determine the optimal function. When the second calibration data does not exist, the determining module performs calibration by using the first calibration data to determine the optimal function. If neither the first calibration data nor the second calibration data exists, in this case, the user is prompted to perform manual calibration.

The calculation module 14 is configured to obtain a current pulse wave transmission time of the user, and calculate a current blood pressure value of the user according to the current pulse wave transmission time and the optimal function.

Further referring to FIG. 17, in an embodiment, the calculation module in the cuff-less blood pressure measurement apparatus in the foregoing embodiment includes a first calculation unit 151 and a second calculation unit 152.

The first calculation unit 151 is configured to obtain a current electrocardiosignal and a current pulse wave signal generated when the user currently performs measurement by using the cuff-less blood pressure measurement apparatus, and calculate the current pulse wave transmission time.

The second calculation unit 152 is configured to calculate the current blood pressure value of the user according to the optimal function and the current pulse wave transmission time.

The determined optimal function is used to represent the function relationship between the pulse wave transmission time and the blood pressure value of the user. Therefore, when the user currently performs measurement by using the cuff-less blood pressure measurement apparatus, the current electrocardiosignal of the user is collected by using the electrocardiography sensor of the cuff-less blood pressure measurement apparatus, and the current pulse wave signal of the user is collected by using at least one of the light sensor, the pressure sensor, the sound sensor, the photoelectric sensor, the acceleration sensor, or the displacement sensor of the cuff-less blood pressure measurement apparatus. The calculation module 14 calculates the current pulse wave transmission time according to the current electrocardiosignal and the current pulse wave signal of the user. Further, the current blood pressure value of the user can be calculated with reference to the determined optimal function according to the calculated current pulse wave transmission time of the user.

In the foregoing embodiment of the present disclosure, calculating systolic blood pressure according to a formula SBP=a1×PTT+b1, and calculating diastolic blood pressure according to a formula DBP=a2×PTT+b2 is merely an implementation example of the present disclosure. Blood pressure may be calculated by using other formulas, for example, the formulas include but are not limited to the following formulas:

$$\begin{matrix}BP = {A}_{1} ln ( {PTT}_{R} ) + {B}_{1} & ( 1 ) \\ BP = {A}_{2} ln ( \frac{{C}_{2}}{{PTT}_{R}^{2}} + 1 ) + {B}_{2} & ( 2 ) \\ BP = \frac{{A}_{3}}{{PTT}_{R}^{2}} + {B}_{3} & ( 3 ) \\ BP = {A}_{4} {PTT}_{R} + {B}_{4} & ( 4 ) \\ BP = {A}_{5} \frac{1}{{PTT}_{R}} + {B}_{5} & ( 5 ) \\ BP = {A}_{6} ( \frac{RT}{{PTT}_{R}^{2}} ) + {B}_{6} . & ( 6 )\end{matrix}$$

In the foregoing formulas, A2=μ×ln(PTT_{w0})
B2=−(SBP₀−DBP₀)×PTT_{w0}/3
C2=SBP₀/3+2DBP₀/3
D2=(SBP₀−DBP₀)×PTT_{w0}²

In the formulas, SBP is systolic blood pressure, DBP is diastolic blood pressure, μ is a vascular characteristic parameter and usually taken as a constant, and a subscript o indicates a calibration value.

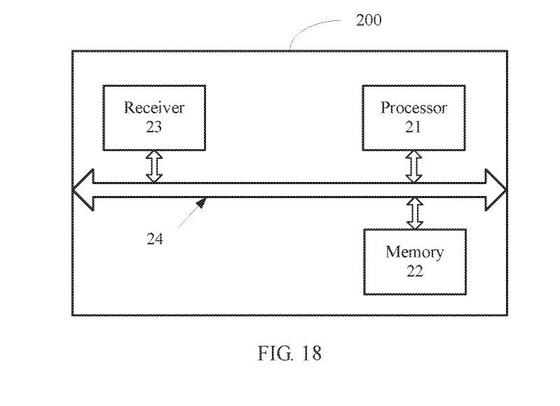

Further referring to FIG. 18, FIG. 18 is a schematic structural diagram of another cuff-less blood pressure measurement apparatus according to an embodiment of the present disclosure. A base station provided in this embodiment is configured to perform a paging method in the embodiment shown in FIG. 1. A cuff-less blood pressure measurement apparatus 200 in this embodiment includes a processor 21, a memory 22, a receiver 23, and a bus system 24.

The processor 21 controls operations of the cuff-less blood pressure measurement apparatus 200, and the processor 21 may also be referred to as a central processing unit (CPU). The processor 21 may be an integrated circuit chip and has a signal processing capability. The processor 21 may be a general purpose processor, a digital signal processor (DSP), an application-specific integrated circuit (ASIC), a field programmable gate array (FPGA) or another programmable logic component, a discrete gate or a transistor logic component, or a discrete hardware component. The general purpose processor may be a microprocessor, or the processor may be any conventional processor or the like.

The memory 22 may include a read-only memory and a random access memory and provide instructions and data for the processor 21. A part of the memory 22 may further include a nonvolatile random access memory (NVRAM).

All components of the cuff-less blood pressure measurement apparatus 200 are coupled together by using the bus system 24. In addition to a data bus, the bus system 24 may include a power supply bus, a control bus, a status signal bus, and the like. The bus system may be an Industry Standard Architecture (ISA) bus, a Peripheral Component Interconnect (PCI) bus, an Extended Industry Standard Architecture (EISA) bus, or the like. The bus may be one or more physical lines. When the bus is multiple physical lines, the bus may be classified into an address bus, a data bus, a control bus, and the like. In some other embodiments of the present disclosure, the processor 21, the memory 22, and the receiver 23 may be directly connected by using a communications line. However, for clear description, various types of buses in the figure are marked as the bus system 24.

The memory 22 stores the following elements in an executable module or data structure, a subset thereof, or an extended set thereof operation instructions, including various operation instructions, and used to implement various operations, and an operating system, including various system programs, and configured to implement various basic services and process hardware-based tasks.

In this embodiment of the present disclosure, the processor 21 executes the following operations by invoking the operation instructions stored in the memory 22 (the operation instructions may be stored in the operating system).

The processor 21 is configured to control the receiver 23 to receive first calibration data of a user, where the first calibration data is data generated when the user performs a manual calibration process before measuring blood pressure by using the cuff-less blood pressure measurement apparatus.

The processor 21 is configured to obtain pre-stored second calibration data of the user; determine, according to the first calibration data and the second calibration data, an optimal function used to represent a relationship between a pulse wave transmission time and a blood pressure value of the user; and further obtain a current pulse wave transmission time of the user, and calculate a current blood pressure value of the user according to the current pulse wave transmission time and the optimal function.

The memory 22 is configured to store the first calibration data and the second calibration data.

The first calibration data includes at least a first blood pressure value and a first pulse wave transmission time.

The manual calibration process described above is as follows. The user obtains the first blood pressure value by performing measurement by using a cuff sphygmomanometer. A first electrocardiosignal of the user is collected by using an electrocardiography sensor of the cuff-less blood pressure measurement apparatus, and a first pulse wave signal of the user is collected by using at least one of a light sensor, a pressure sensor, a sound sensor, a photoelectric sensor, an acceleration sensor, or a displacement sensor of the cuff-less blood pressure measurement apparatus. The first pulse wave transmission time is calculated according to the first electrocardiosignal and the first pulse wave signal of the user. Each time the user performs manual calibration, a group of a first blood pressure value and a first pulse wave transmission time is generated. The processor 21 controls the receiver 23 to receive the first blood pressure value and the first pulse wave transmission time that are manually input by the user or obtain the first blood pressure value and the first pulse wave transmission time from a cuff blood pressure measurement apparatus by using a specific interface such as Bluetooth or an infrared interface, so as to form the first calibration data.

In a possible implementation, calculating the first pulse wave transmission time according to the first electrocardiosignal and the first pulse wave signal may correspond to calculating the first pulse wave transmission time according to a time difference between a reference point on the first electrocardiosignal and a reference point on the first pulse wave signal in a same period as the reference point on the first electrocardiosignal.

The second calibration data includes at least a second blood pressure value and a second pulse wave transmission time. The second calibration data may be historical manual calibration data generated before the first calibration data is obtained and when the user performs a manual calibration process by using the cuff-less blood pressure measurement apparatus. The historical manual calibration data is stored in the cuff-less blood pressure measurement apparatus. Alternatively, the second calibration data may be calibration data pre-stored by the user on a cloud side. The calibration data stored on the cloud side may be calibration data exported from an intelligent wearable device such as a wristwatch. The processor 21 obtains, from the cloud side by using an internal interface, the calibration data stored on the cloud side, and uses the calibration data as the second calibration data.

The processor 21 may obtain an identity of the user, and obtain the second calibration data from multiple pieces of pre-stored calibration data according to the identity of the user. The processor 21 may determine the identity of the user according to one or two of a first electrocardiosignal or a first pulse wave signal in the first calibration data of the user, or determine the identity of the user according to one or two of a current electrocardiosignal or a current pulse wave signal generated when the user currently performs measurement by using the cuff-less blood pressure measurement apparatus.

In this embodiment of the present disclosure, the processor 21 determines an optimal function used to represent a function relationship between a pulse wave transmission time and a blood pressure value of the user by determining a calibration parameter (or a calibration coefficient) for calculating the blood pressure value according to the pulse wave transmission time. Specifically, for example, systolic blood pressure is calculated by using a formula SBP=a1×PTT+b1, and diastolic blood pressure is calculated according to a formula DBP=a2×PTT+b2. Determining the optimal function in this embodiment of the present disclosure corresponds to determining values of a1, b1, a2, and b2 in the formulas, and the blood pressure value of the user is accordingly calculated according to current measurement data and the determined calibration parameter.

The determined optimal function is used to represent the function relationship between the pulse wave transmission time and the blood pressure value of the user. Therefore, when the user currently performs measurement by using the cuff-less blood pressure measurement apparatus, the current electrocardiosignal of the user is collected by using the electrocardiography sensor of the cuff-less blood pressure measurement apparatus, and the current pulse wave signal of the user is collected by using at least one of the light sensor, the pressure sensor, the sound sensor, the photoelectric sensor, the acceleration sensor, or the displacement sensor of the cuff-less blood pressure measurement apparatus. The current pulse wave transmission time is calculated according to the current electrocardiosignal and the current pulse wave signal of the user. The current blood pressure value of the user can be calculated with reference to the determined optimal function according to the calculated current pulse wave transmission time of the user.

In the foregoing embodiment of the present disclosure, calculating systolic blood pressure according to a formula SBP=a1×PTT+b1, and calculating diastolic blood pressure according to a formula DBP=a2×PTT+b2 is merely an implementation example of the present disclosure. Blood pressure may be calculated by using other formulas, for example, the formulas include but are not limited to the following formulas:

$$\begin{matrix}BP = {A}_{1} ln ( {PTT}_{R} ) + {B}_{1} & ( 1 ) \\ BP = {A}_{2} ln ( \frac{{C}_{2}}{{PTT}_{R}^{2}} + 1 ) + {B}_{2} & ( 2 ) \\ BP = \frac{{A}_{3}}{{PTT}_{R}^{2}} + {B}_{3} & ( 3 ) \\ BP = {A}_{4} {PTT}_{R} + {B}_{4} & ( 4 ) \\ BP = {A}_{5} \frac{1}{{PTT}_{R}} + {B}_{5} & ( 5 ) \\ BP = {A}_{6} ( \frac{RT}{{PTT}_{R}^{2}} ) + {B}_{6} . & ( 6 )\end{matrix}$$

In the foregoing formulas, A2=μ×ln(PTT_{w0})
B2=−(SBP₀−DBP₀)×PTT_{w0}/3
C2=SBP₀/3+2DBP₀/3
D2=(SBP₀−DBP₀)×PTT_{w0}²

In the formulas, SBP is systolic blood pressure, DBP is diastolic blood pressure, μ is a vascular characteristic parameter and usually taken as a constant, and a subscript o indicates a calibration value.

The processor 21 may determine the optimal function by determining a first function according to the first calibration data, determining a second function according to the second calibration data, determining a degree of difference between the first function and the second function, and determining the optimal function according to the degree of difference.

The first function herein is a function used to represent a relationship between a first blood pressure value and a first pulse wave transmission time in the first calibration data. The second function herein is a function used to represent a relationship between a second blood pressure value and a second pulse wave transmission time in the second calibration data.

In a possible implementation solution, the processor 21 determines the first function according to the first calibration data by using a least square method. The processor 21 determines the second function according to the second calibration data by using the least square method.

The degree of difference between the first function and the second function may be measured by using a linear relationship for associating the two functions in a same coordinate system. Specifically, after the first function and the second function are determined by using the least square method, a slope change rate and/or a fitting coefficient change rate of the first function relative to the second function are/is determined to determine the degree of difference.

After the degree of difference between the two functions is determined, the optimal function used to represent the relationship between the pulse wave transmission time and the blood pressure value of the user is determined according to the degree of difference.

In an implementation, if the degree of difference is less than a first preset threshold, a third function determined by using the first calibration data and the second calibration data is used as the optimal function. The second function is used as the optimal function if the degree of difference is greater than the first preset threshold and a quantity of samples of the first calibration data is less than a second preset threshold. Alternatively, the first function is used as the optimal function if the degree of difference is greater than the first preset threshold and a quantity of samples of the first calibration data is greater than a second preset threshold. The first preset threshold and the second preset threshold herein may be thresholds that are preset and stored by the user in the cuff-less blood pressure measurement apparatus. The user may adjust the first preset threshold and the second preset threshold according to a requirement. When the degree of difference is indicated by using the slope change rate and the fitting coefficient change rate of the first function relative to the second function, the first preset threshold correspondingly includes two thresholds (which are respectively a slope change rate threshold and a fitting coefficient change rate threshold). For example, the slope change rate threshold is 30%, and the fitting system change rate threshold is 10%. In addition, the second preset threshold is for the quantity of samples that may be set to, for example, 4 or 6.

That is, when the degree of difference between the first function and the second function is less than the first preset threshold, it may be determined that a deviation between the first calibration data and the pre-stored second calibration data is not large, and the function determined by using a combination of the two pieces of calibration data may be used as the optimal function. If the degree of difference is greater than the first preset threshold, it is determined that a deviation between the first calibration data and the pre-stored second calibration data is relatively large. In this case, determining needs to be further performed with reference to the quantity of samples of the first calibration data. If the quantity of samples of the first calibration data is large enough (exceeds the second preset threshold), the first function determined by using the first calibration data may be independently used as the optimal function. If the quantity of samples of the first calibration data is relatively small (does not exceed the second preset threshold), the second function determined by using the pre-stored second calibration data is directly used as the optimal function.

In addition, other implementations may be as follows. If the degree of difference is less than a first preset threshold, a third function determined by using the first calibration data and the second calibration data is used as the optimal function. If the degree of difference is greater than the first preset threshold and a quantity of samples of the first calibration data is less than a second preset threshold, the third function determined by using the first calibration data and the second calibration data is used as the optimal function. Alternatively, if the degree of difference is greater than the first preset threshold and a quantity of samples of the first calibration data is greater than a second preset threshold, an abnormal data point is removed from the first calibration data, and a fourth function calculated by using a combination of the second calibration data and remaining first calibration data is used as the optimal function.

In an implementation, when the degree of difference between the first function and the second function is less than the first preset threshold, it may be determined that a deviation between the first calibration data and the pre-stored second calibration data is not large, and the function determined by using a combination of the two pieces of calibration data may be used as the optimal function. When the degree of difference is greater than the first preset threshold and the quantity of samples of the first calibration data is less than the second preset threshold, because a quantity of samples of the second calibration data is small, a difference brought by the second calibration data may be ignored, and the third function determined by using the combination of the first calibration data and the second calibration data is used as the optimal function. If the degree of difference is greater than the first preset threshold and the quantity of samples of the first calibration data is greater than the second preset threshold, a deviation of the first calibration data relative to the pre-stored second calibration data is relatively large, and the quantity of samples of the first calibration data is relatively large. In this case, residual analysis may be further performed on the first calibration data to determine whether there is an abnormal point in the first calibration data. If there is an abnormal point, after the abnormal point is removed, the fourth function calculated by using the combination of the second calibration data and the remaining first calibration data is used as the optimal function.

Alternatively, the processor 21 may determine the optimal function by determining a first function according to the first calibration data, determining a third function according to a combination of the first calibration data and the second calibration data, determining a degree of difference between the first function and the third function, and determining the optimal function according to the degree of difference.

The first function herein is a function used to represent a relationship between a first blood pressure value and a first pulse wave transmission time in the first calibration data. The third function herein is a function used to represent a relationship between a blood pressure value and a pulse wave transmission time in the combination of the first calibration data and the second calibration data.

In a possible implementation solution, the processor 21 determines the first function according to the first calibration data by using a least square method. The processor 21 determines the third function according to the first calibration data and the second calibration data by using the least square method.

The degree of difference between the first function and the third function may be measured by using a linear relationship for associating the two functions in a same coordinate system. Specifically, after the first function and the third function are determined by using the least square method, a slope change rate and/or a fitting coefficient change rate of the third function relative to a second function are/is determined to determine the degree of difference.

After the degree of difference between the two functions is determined, the optimal function used to represent the relationship between the pulse wave transmission time and the blood pressure value of the user is determined according to the degree of difference.

In an implementation, if the degree of difference between the first function and the third function is less than a first preset threshold, the third function determined by using the first calibration data and the second calibration data is used as the optimal function. A second function is used as the optimal function if the degree of difference is greater than the first preset threshold and a quantity of samples of the first calibration data is less than a second preset threshold. Alternatively, the first function is used as the optimal function if the degree of difference is greater than the first preset threshold and a quantity of samples of the first calibration data is greater than a second preset threshold. The first preset threshold and the second preset threshold herein may be thresholds that are preset and stored by the user in the cuff-less blood pressure measurement apparatus. The user may adjust the first preset threshold and the second preset threshold according to a requirement. When the degree of difference is indicated by using the slope change rate and the fitting coefficient change rate of the first function relative to the second function, the first preset threshold correspondingly includes two thresholds (which are respectively a slope change rate threshold and a fitting coefficient change rate threshold). For example, the slope change rate threshold is 30%, and the fitting system change rate threshold is 10%. In addition, the second preset threshold is for the quantity of samples that may be set to, for example, 4 or 6.

That is, when the degree of difference between the first function and the third function is less than the first preset threshold, it may be determined that a deviation between the first calibration data and the pre-stored second calibration data is not large, and the function determined by using the combination of the two pieces of calibration data may be used as the optimal function. If the degree of difference is greater than the first preset threshold, it is determined that a deviation between the first calibration data and the pre-stored second calibration data is relatively large. In this case, determining needs to be further performed with reference to the quantity of samples of the first calibration data. If the quantity of samples of the first calibration data is large enough (exceeds the second preset threshold), the first function determined by using the first calibration data may be independently used as the optimal function. If the quantity of samples of the first calibration data is relatively small (does not exceed the second preset threshold), the second function determined by using the pre-stored second calibration data is directly used as the optimal function.

In addition, other implementations may be as follows. If the degree of difference between the first function and the third function is less than a first preset threshold, the third function determined by using the first calibration data and the second calibration data is used as the optimal function. If the degree of difference is greater than the first preset threshold and a quantity of samples of the first calibration data is less than a second preset threshold, the third function determined by using the first calibration data and the second calibration data is used as the optimal function. Alternatively, if the degree of difference is greater than the first preset threshold and a quantity of samples of the first calibration data is greater than a second preset threshold, an abnormal data point is removed from the first calibration data, and a fourth function calculated by using a combination of the second calibration data and remaining first calibration data is used as the optimal function.

In an implementation, when the degree of difference between the first function and the third function is less than the first preset threshold, it may be determined that a deviation between the first calibration data and the pre-stored second calibration data is not large, and the function determined by using the combination of the two pieces of calibration data may be used as the optimal function. When the degree of difference is greater than the first preset threshold and the quantity of samples of the first calibration data is less than the second preset threshold, because a quantity of samples of the second calibration data is small, a difference brought by the second calibration data may be ignored, and the third function determined by using the combination of the first calibration data and the second calibration data is used as the optimal function. If the degree of difference is greater than the first preset threshold and the quantity of samples of the first calibration data is greater than the second preset threshold, a deviation of the first calibration data relative to the pre-stored second calibration data is relatively large, and the quantity of samples of the first calibration data is relatively large. In this case, residual analysis may be further performed on the first calibration data to determine whether there is an abnormal point in the first calibration data. If there is an abnormal point, after the abnormal point is removed, the fourth function calculated by using the combination of the second calibration data and the remaining first calibration data is used as the optimal function.

Alternatively, the processor 21 may determine the optimal function by obtaining a current pulse wave transmission time of the user; selecting, from the first calibration data and the second calibration data, a pulse wave transmission time closest to the current pulse wave transmission time; using calibration data in which the pulse wave transmission time closest to the current pulse wave transmission time exists as optimal calibration data; and using a function as the optimal function, where the function is determined according to the optimal calibration data.

The current pulse wave transmission time of the user may be obtained in the following manner. A corresponding electrocardiosignal and a corresponding pulse wave signal generated when the user currently uses the cuff-less blood pressure measurement apparatus are obtained, and the current pulse wave transmission time of the user is calculated. A current electrocardiosignal of the user is collected by using the electrocardiography sensor of the cuff-less blood pressure measurement apparatus, and a current pulse wave signal of the user is collected by using at least one of the light sensor, the pressure sensor, the sound sensor, the photoelectric sensor, the acceleration sensor, or the displacement sensor of the cuff-less blood pressure measurement apparatus. The current pulse wave transmission time of the user is calculated according to the current electrocardiosignal and the current pulse wave signal of the user.

The obtained current pulse wave transmission time of the user is referred to as PTT3. The current pulse wave transmission time PTT3 is separately compared with a first pulse wave transmission time PTT1 in the first calibration data and a second pulse wave transmission time PTT2 in the second calibration data, to determine whether a difference value between the PTT1 and the PTT3 or a difference value between the PTT2 and the PTT3 is smaller. If the difference value between the PTT1 and the PTT3 is smaller, the optimal function is determined by using the first calibration data. If the difference value between the PTT2 and the PTT3 is smaller, the optimal function is determined by using the second calibration data. That is, in this implementation, with reference to the PPT1 in the first calibration data, the PTT2 in the second calibration data, and the PTT3 calculated by using data obtained by means of current measurement, calibration data corresponding to a PTT closest to the PTT3 is used as the optimal calibration data, and a function determined by using the optimal calibration data is used as the optimal function.

Herein, when specific comparison is performed, an average value of pulse wave transmission times calculated by using multiple pairs of data in the first calibration data may be calculated and used as the first pulse wave transmission time PTT1, an average value of pulse wave transmission times calculated by using multiple pairs of data in the second calibration data may be calculated and used as the second pulse wave transmission time PTT2, and the PTT1 and the PTT2 are separately compared with the PTT3 to determine the optimal calibration data.

Alternatively, a PTT closest to the PTT3 may be found in multiple first pulse wave transmission times PTT1 in the first calibration data and multiple second pulse wave transmission times PTT2 in the second calibration data, and calibration data in which the PTT closest to the PTT3 exists is used as the optimal calibration data. For example, there are multiple first pulse wave transmission times A, B, C, and D in the first calibration data, and there are multiple second pulse wave transmission times A1, B1, C1, and D1 in the second calibration data. If one of A, B, C, or D is closest to the PTT3, the first calibration data is used as the optimal calibration data. If one of A1, B1, C1, or D1 is closest to the PTT3, the second calibration data is used as the optimal calibration data.

After the optimal calibration data is determined, the function determined according to the optimal calibration data by using a least square method is used as the optimal function used to represent the relationship between the pulse wave transmission time and the blood pressure value of the user.

The method disclosed in the foregoing embodiment of the present disclosure may be applied to the processor 21, or implemented by the processor 21. In an implementation process, the steps in the foregoing method may be completed by using an integrated logic circuit of hardware in the processor 21 or an instruction in a form of software. The methods, steps, and logical block diagrams disclosed in the embodiments of the present disclosure may be implemented or executed. The steps of the methods disclosed with reference to the embodiments of the present disclosure may be directly executed and completed by means of a hardware decoding processor, or may be executed and completed by using a combination of hardware and software modules in the decoding processor. The software module may be located in a mature storage medium in the art, such as a random access memory, a flash memory, a read-only memory, a programmable read-only memory, an electrically-erasable programmable memory, or a register. The storage medium is located in the memory 22. The processor 21 reads information in the memory 22, and completes the steps of the foregoing methods in combination with the hardware of the processor 21.

According to detailed description of the blood pressure measurement data processing method and the cuff-less blood pressure measurement apparatus provided in the embodiments of the present disclosure, it can be understood that in the present disclosure, first calibration data generated when a user performs manual calibration and pre-stored second calibration data are combined, and an optimal function used to represent a function relationship between a pulse wave transmission time and a blood pressure value of the user is determined according to the first calibration data and the second calibration data. In such a manner, when a user wears a cuff-less blood pressure measurement apparatus for measurement, automatic calibration can be performed with reference to pre-stored calibration data to determine an optimal function, so that the pre-stored calibration data can be fully used, and calibration is more accurate. Therefore, a measurement result of a blood pressure value is more accurate.

In the several embodiments provided in this application, it should be understood that the disclosed systems, apparatuses, and methods may be implemented in other manners. For example, the described apparatus embodiments are merely examples. For example, the module or unit division is merely logical function division and may be other division in actual implementation. For example, multiple units or components may be combined or integrated into another system, or some features may be ignored or not performed. In addition, the displayed or discussed mutual couplings or direct couplings or communication connections may be implemented by using some interfaces. The indirect couplings or communication connections between the apparatuses or units may be implemented in electronic, mechanical, or other forms.

The units described as separate parts may or may not be physically separate, and parts displayed as units may or may not be physical units, may be located in one position, or may be distributed on multiple network units. Some or all of the units may be selected according to actual needs to achieve the objectives of the solutions of the embodiments.

In addition, functional units in the embodiments of this application may be integrated into one processing unit, or each of the units may exist alone physically, or two or more units are integrated into one unit. The integrated unit may be implemented in a form of hardware, or may be implemented in a form of a software functional unit.

When the integrated unit is implemented in the form of a software functional unit and sold or used as an independent product, the integrated unit may be stored in a computer-readable storage medium. Based on such an understanding, the technical solutions of this application essentially, or the part contributing to the prior art, or all or some of the technical solutions may be implemented in the form of a software product. The computer software product is stored in a storage medium and includes several instructions for instructing a computer device (which may be a personal computer, a server, a network device, or the like) or a processor to perform all or some of the steps of the methods described in the embodiments of this application. The foregoing storage medium includes any medium that can store program code, such as a USB flash drive, a removable hard disk, a read-only memory (ROM), a random access memory (RAM), a magnetic disk, or an optical disc.

The foregoing descriptions are merely embodiments of this application, and are not intended to limit the scope of this application. All equivalent structural or equivalent process changes made by using the content of the specification and accompanying drawings of this application or by directly or indirectly applying this application to other related technical fields shall fall within the protection scope of this application.

1. A blood pressure measurement data processing method, comprising:
obtaining, by a cuff-less blood pressure measurement apparatus, first calibration data of a user, wherein the first calibration data is data generated when the user performs a manual calibration process before measuring blood pressure using the cuff-less blood pressure measurement apparatus;
obtaining pre-stored second calibration data of the user;
determining, according to the first calibration data and the second calibration data, an optimal function used to represent a relationship between a pulse wave transmission time and a blood pressure value of the user;
obtaining a current pulse wave transmission time of the user; and
calculating a current blood pressure value of the user according to the current pulse wave transmission time and the optimal function.

2. The method according to claim 1, wherein the determining, according to the first calibration data and the second calibration data, an optimal function used to represent a relationship between a pulse wave transmission time and a blood pressure value of the user comprises:
determining a first function according to the first calibration data;
determining a second function according to the second calibration data;
determining a degree of difference between the first function and the second function; and
determining the optimal function according to the degree of difference.

3. The method according to claim 2, wherein the determining a first function according to the first calibration data comprises determining the first function according to the first calibration data by using a least square method, and wherein the determining a second function according to the second calibration data comprises determining the second function according to the second calibration data by using the least square method.

4. The method according to claim 2, wherein the determining the optimal function according to the degree of difference comprises using a third function as the optimal function when the degree of difference is less than a first preset threshold, and wherein the third function is determined by using the first calibration data and the second calibration data.

5. The method according to claim 4, wherein the determining the optimal function according to the degree of difference further comprises:
using the second function as the optimal function when the degree of difference is greater than the first preset threshold and a quantity of samples of the first calibration data is less than a second preset threshold; or
using the first function as the optimal function when the degree of difference is greater than the first preset threshold and a quantity of samples of the first calibration data is greater than the second preset threshold.

6. The method according to claim 4, wherein the determining the optimal function according to the degree of difference further comprises:
when the degree of difference is greater than the first preset threshold and a quantity of samples of the first calibration data is less than a second preset threshold, using the third function as the optimal function, wherein the third function is determined by using a combination of the first calibration data and the second calibration data; or
when the degree of difference is greater than the first preset threshold and a quantity of samples of the first calibration data is greater than the second preset threshold, removing an abnormal data point from the first calibration data, and using a fourth function as the optimal function, wherein the fourth function is calculated by using a combination of the second calibration data and remaining first calibration data.

7. The method according to claim 1, wherein the determining, according to the first calibration data and the second calibration data, an optimal function of a relationship between a pulse wave transmission time and a blood pressure value of the user comprises:
determining a first function according to the first calibration data;
determining a third function according to a combination of the first calibration data and the second calibration data;
determining a degree of difference between the first function and the third function; and
determining the optimal function according to the degree of difference.

8. The method according to claim 7, wherein the determining a first function according to the first calibration data comprises determining the first function according to the first calibration data by using a least square method, and wherein the determining a third function according to a combination of the first calibration data and the second calibration data comprises determining the third function according to the combination of the first calibration data and the second calibration data by using the least square method.

9. The method according to claim 7, wherein the determining the optimal function according to the degree of difference comprises using the third function as the optimal function when the degree of difference is less than a first preset threshold.

10. The method according to claim 9, wherein the determining the optimal function according to the degree of difference further comprises:
when the degree of difference is greater than the first preset threshold and a quantity of samples of the first calibration data is less than a second preset threshold, using a second function as the optimal function, wherein the second function is determined by using the second calibration data; or
using the first function as the optimal function when the degree of difference is greater than the first preset threshold and a quantity of samples of the first calibration data is greater than the second preset threshold.

11. The method according to claim 9, wherein the determining the optimal function according to the degree of difference further comprises:
using the third function as the optimal function when the degree of difference is greater than the first preset threshold and a quantity of samples of the first calibration data is less than a second preset threshold; or
when the degree of difference is greater than the first preset threshold and a quantity of samples of the first calibration data is greater than the second preset threshold, removing an abnormal data point from the first calibration data, and using a fourth function as the optimal function, wherein the fourth function is calculated by using a combination of the second calibration data and remaining first calibration data.

12. The method according to claim 1, wherein the first calibration data and the second calibration data separately comprise at least one group of a blood pressure value and a corresponding pulse wave transmission time, and wherein the determining, according to the first calibration data and the second calibration data, an optimal function used to represent a relationship between a pulse wave transmission time and a blood pressure value of the user comprises:
obtaining a current pulse wave transmission time of the user;
selecting, from the first calibration data and the second calibration data, a pulse wave transmission time closest to the current pulse wave transmission time;
using calibration data in which the pulse wave transmission time closest to the current pulse wave transmission time exists as optimal calibration data; and
using a function as the optimal function, wherein the function is determined according to the optimal calibration data.

13. The method according to claim 1, wherein the obtaining pre-stored second calibration data of the user comprises:
obtaining, by the cuff-less blood pressure measurement apparatus, an identity of the user; and
obtaining the second calibration data from multiple pieces of pre-stored calibration data according to the identity of the user.

14. The method according to claim 13, wherein the obtaining an identity of the user comprises:
determining the identity of the user according to at least one of a first electrocardiosignal or a first pulse wave signal in the first calibration data of the user; or
determining the identity of the user according to at least one of a current electrocardiosignal or a current pulse wave signal generated when the user currently performs measurement by using the cuff-less blood pressure measurement apparatus.

15. The method according to claim 1, wherein the obtaining a current pulse wave transmission time of the user, and calculating a current blood pressure value of the user according to the current pulse wave transmission time and the optimal function comprises:
obtaining a current electrocardiosignal and a current pulse wave signal generated when the user currently performs measurement by using the cuff-less blood pressure measurement apparatus, and calculating the current pulse wave transmission time; and
calculating the current blood pressure value of the user according to the optimal function and the current pulse wave transmission time.

16.-45. (canceled)

46. A cuff-less blood pressure measurement apparatus comprising:
at least one processor; and
at least one memory comprising instructions that, when executed by the at least one processor, cause the cuff-less blood pressure measurement apparatus to:
obtain first calibration data of a user, wherein the first calibration data is data generated when the user performs a manual calibration process before measuring blood pressure using the cuff-less blood pressure measurement apparatus;
obtain pre-stored second calibration data of the user;
determine, according to the first calibration data and the second calibration data, an optimal function used to represent a relationship between a pulse wave transmission time and a blood pressure value of the user;
obtain a current pulse wave transmission time of the user; and
calculate a current blood pressure value of the user according to the current pulse wave transmission time and the optimal function.

47. The cuff-less blood pressure measurement apparatus according to claim 46, wherein the instructions, when executed by the at least one processor, cause the cuff-less blood pressure measurement apparatus to:
determine a first function according to the first calibration data;
determine a second function according to the second calibration data;
determine a degree of difference between the first function and the second function; and
determine the optimal function according to the degree of difference.

48. The cuff-less blood pressure measurement apparatus according to claim 47, wherein the instructions, when executed by the at least one processor, cause the cuff-less blood pressure measurement apparatus to:
determine the first function according to the first calibration data by using a least square method; and
determine the second function according to the second calibration data by using the least square method.

49. The cuff-less blood pressure measurement apparatus according to claim 47, wherein the instructions, when executed by the at least one processor, cause the cuff-less blood pressure measurement apparatus to use a third function as the optimal function when the degree of difference is less than a first preset threshold, wherein the third function is determined by using the first calibration data and the second calibration data.

50. The cuff-less blood pressure measurement apparatus according to claim 49, wherein the instructions, when executed by the at least one processor, cause the cuff-less blood pressure measurement apparatus to:
use the second function as the optimal function when the degree of difference is greater than the first preset threshold and a quantity of samples of the first calibration data is less than a second preset threshold; or
use the first function as the optimal function when the degree of difference is greater than the first preset threshold and a quantity of samples of the first calibration data is greater than the second preset threshold.
